(19) United States
(12) Patent Application Publication
(10) Pub. No.: US 2007/0072906 A1
(43) Pub. Date: Mar. 29, 2007

(54) Pyrrole compounds

(76) Inventors: Gerard Martin Paul Giblin, Essex (GB); Adrian Hall, Essex (GB); Mark Patrick Healy, Essex (GB); Francis David King, Essex (GB); Xiao Qing Lewell, Hertfordshire (GB); Neil Derek Miller, Essex (GB); Alan Naylor, Essex (GB); Riccardo Novelli, Essex (GB)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 10/596,153

(22) PCT Filed: Nov. 30, 2004
(86) PCT No.: PCT/EP04/13744

§ 371 Date: Sep. 13, 2006

(30) Foreign Application Priority Data
Dec. 3, 2003 (GB) 0328024.5

(51) Int. Cl. A61K 31/454; A61K 31/41; A61K 31/4025; C07D 403/02

(52) U.S. Cl. 514/326; 514/381; 514/422; 546/208; 548/253; 548/518

(57) ABSTRACT

Compounds of formula (I) or a derivative thereof: 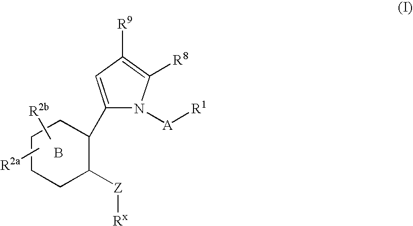
wherein A, B, Z, R¹, R^{2a}, R^{2b}, R^{x}, R⁶, and R⁹ are as defined in the specification, a process for the preparation of such compounds, pharmaceutical compositions comprising such compounds and the use of such compounds in medicine.

This invention relates to pyrrole derivatives, to processes for their preparation, to pharmaceutical compositions containing them and to their use in medicine, in particular their use in the treatment of conditions mediated by the action of PGE₂ at the EP₁ receptor.

The EP₁ receptor is a 7-transmembrane receptor and its natural ligand is the prostaglandin PGE₂. PGE₂ also has affinity for the other EP receptors (types EP₂, EP₃ and EP₄). The EP₁ receptor is associated with smooth muscle contraction, pain (in particular inflammatory, neuropathic and visceral), inflammation, allergic activities, renal regulation and gastric or enteric mucus secretion. We have now found a novel group of compounds which bind with high affinity to the EP₁ receptor.

A number of review articles describe the characterization and therapeutic relevance of the prostanoid receptors as well as the most commonly used selective agonists and antagonists: Eicosanoids; From Biotechnology to Therapeutic Applications, Folco, Samuelsson, Maclouf, and Velo eds, Plenum Press, New York, 1996, chap. 14, 137-154 and Journal of Lipid Mediators and Cell Signalling, 1996, 14, 83-87 and Prostanoid Receptors, Structure, Properties and Function, S Narumiya et al, Physiological Reviews 1999, 79(4), 1193-126. An article from The British Journal of Pharmacology (1994, 112, 735-740) suggests that Prostaglandin E₂ (PGE₂) exerts allodynia through the EP₁ receptor subtype and hyperalgesia through EP₂ and EP₃ receptors in the mouse spinal cord. Furthermore an article from The Journal of Clinical Investigation (2001, 107 (3), 325) shows that in the EP₁ knock-out mouse pain-sensitivity responses are reduced by approximately 50%. Two papers from Anesthesia and Analgesia have shown that (2001, 93, 1012-7) an EP₁ receptor antagonist (ONO-8711) reduces hyperalgesia and allodynia in a rat model of chronic constriction injury, and that (2001, 92, 233-238) the same antagonist inhibits mechanical hyperalgesia in a rodent model of post-operative pain. S. Sarkar et al in Gastroenterology, 2003, 124(1), 18-25 demonstrate the efficacy of EP₁ receptor antagonists in the treatment of visceral pain in a human model of hypersensitivity. Thus, selective prostaglandin ligands, agonists or antagonists, depending on which prostaglandin E receptor subtype is being considered, have ant-inflammatory, antipyretic and analgesic properties similar to a conventional non-steroidal anti-inflammatory drug, and in addition, inhibit hormone-induced uterine contractions and have ant-cancer effects. These compounds have a diminished ability to induce some of the mechanism-based side effects of NSAIDs which are indiscriminate cyclooxygenase inhibitors. In particular, the compounds have a reduced potential for gastrointestinal toxicity, a reduced potential for renal side effects, a reduced effect on bleeding times and a lessened ability to induce asthma attacks in aspirin-sensitive asthmatic subjects. Moreover, by sparing potentially beneficial prostaglandin pathways, these agents may have enhanced efficacy over NSAIDS and/or COX-2 inhibitors.

In The American Physiological Society (1994, 267, R289-R-294), studies suggest that PGE₂-induced hyperthermia in the rat is mediated predominantly through the EP₁ receptor.

WO 96/06822 (Mar. 7, 1996), WO 96/11902 (Apr. 25, 1996), EP 752421 -A1 (Jan. 08, 1997), WO 01/19814 (22 Mar. 2001), WO 03/084917 (16 Oct. 2003), WO 03/101959 (11 Dec. 2003), WO 2004/039753 (13 May 2004) and WO 2004/083185 (30 Sep. 2004) disclose compounds as being useful in the treatment of prostaglandin mediated diseases.

P. Lacombe et al (220th National Meeting of The American Chemical Society, Washington D.C., USA, 20-24 Aug., 2000) disclosed 2,3-diarylthiophenes as ligands for the human EP₁ prostanoid receptor. Y. Ducharme et al (18^{th} International Symposium on Medicinal Chemistry; Copenhagen, Denmark and Malmo, Sweden; 15-19 Aug. 2004) disclosed 2,3-diarylthiophenes as EP₁ receptor antagonists.

It is now indicated that a novel group of pyrrole derivatives are selective for the EP₁ receptor over the EP₃ receptor, and are therefore potentially useful in treating conditions mediated by the action of PGE₂ at EP₁ receptors. Such conditions include inflammatory, immunological, bone, neurodegenerative and renal disorders, and pain.

Accordingly the present invention provides a compound of formula (I): 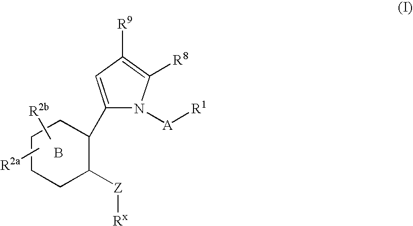
wherein:
A represents an optionally substituted aryl, or an optionally substituted 5- or 6-membered heterocyclyl ring, or an optionally substituted bicyclic heterocyclyl group;
B represents a phenyl or pyridyl ring;
Z represents O, S, SO, or SO₂;
R¹ represents CO₂R⁴, CN, CONR⁵R⁶, CH₂CO₂R⁴, OR⁴, optionally substituted alkyl, optionally substituted alkenyl, optionally substituted SO₂alkyl, SO₂NR⁵R⁶, NR⁵CONR⁵R⁶, COalkyl, 2H-tetrazol-5-yl-methyl, optionally substituted bicyclic heterocycle or optionally substituted heterocyclyl;
R^{2a} and R^{2b} each independently represent hydrogen, halogen, optionally substituted alkyl, optionally substituted alkoxy, CN, SO₂alkyl, SR⁵, NO₂, optionally substituted aryl, CONR⁵R⁶ or optionally substituted heteroaryl;
R^{x} represents optionally substituted alkyl wherein 1 or 2 of the non-terminal carbon atoms are optionally substituted by a group independently selected from NR⁴, O and SOₙ, wherein n is 0, 1 or 2; optionally substituted alkenyl; or optionally substituted alkynyl: or R^{x} represents optionally substituted alkenyl, optionally substituted CQ^{a}Q^{b}-heterocyclyl, optionally substituted CQ^{a}Q^{b}-bicyclic heterocyclyl or optionally substituted CQ^{a}Q^{b}-aryl;
R⁴ represents hydrogen or an optionally substituted alkyl;
R⁵ represents hydrogen or an optionally substituted alkyl;
R⁶ represents hydrogen or optionally substituted alkyl, optionally substituted heteroaryl, optionally substituted SO₂aryl, optionally substituted SO₂alkyl, optionally substituted SO₂heteroaryl, CN, optionally substituted CQ^{a}Q^{b}aryl, optionally substituted CQ^{a}Q^{b}heteroaryl or COR⁷;
R⁷ represents hydrogen, optionally substituted alkyl, optionally substituted heteroaryl or optionally substituted aryl;
R⁸ represents hydrogen, CF₃, Cl or C₁₋₃alkyl;
R⁹ represents halogen, hydrogen, CF₃, or C₁₋₃alkyl;
Q^{a} and Q^{b} are each independently selected from hydrogen and CH₃; wherein when A is a 6-membered ring the R¹ substituent and pyrrole ring are attached to carbon atoms 1,2-, 1,3- or 1,4-relative to each other, and when A is a five-membered ring or bicyclic heterocyclyl group the R¹ substituent and phenyl ring are attached to substitutable carbon atoms 1,2- or 1,3-relative to each other; and derivatives thereof.

Suitably A is optionally substituted phenyl, an optionally substituted 6-membered heterocyclyl ring or an optionally substituted bicyclic heterocyclyl group. More suitably A is optionally substituted phenyl, optionally substituted pyridyl or optionally substituted isoquinolinyl. When A is pyridyl, suitably the R¹ and pyrrole groups are attached to the 2- and 6-positions of the pyridine ring.

Yet more suitably A is selected from phenyl optionally substituted by methyl or fluoro; pyridyl wherein the R¹ and pyrrole groups are attached to the 2- and 6-positions of the pyridine ring; and isoquinolinyl.

Optional substituents for A include up to four substituents, preferably 0 or 1 substituent, independently selected from halogen or optionally substituted C₁₋₃alkyl.

Suitable optional substituents for A include fluoro and CH₃.

In one embodiment A is pyridyl.

In one aspect A is 6-methylbenzoic acid substituted on the 3-position by the pyrrolyl group, or picolinic acid substituted on the 6-position by the pyrrolyl group. In an alternative aspect A is 6-fluorobenzoic acid substituted on the 3-position by the pyrrolyl group.

Suitably B is phenyl.

Suitably Z is O.

Suitably R¹ is CO₂H.

Suitably R^{2a} is hydrogen.

Suitably R^{2b} is selected from hydrogen, halogen, and optionally substituted C₁₋₄alkyl e.g. CF₃. More suitably R^{2b} is Br, Cl or CF₃.

Preferably R^{2b} is positioned 1,4-relative to the Z substituent and 1,3-relative to the pyrrolyl ring.

Suitably R^{x} includes optionally substituted C₃₋₈alkyl, optionally substituted CH₂pyridyl, optionally substituted CH₂thienyl or optionally substituted CH₂phenyl. In one aspect R^{x} represents optionally substituted CH₂phenyl.

Suitable optional substituents for R^{x} when CH₂phenyl include one to three substituents selected from Cl, F, Br, CH₃ and CF₃, particular substituents are selected from Cl, Br and F.

Suitably when R^{x} is optionally substituted alkyl it is preferably C₃₋₈alkyl, for example cyclopentylmethylene or isobutyl.

Suitably R⁴ is hydrogen or C₁₋₄alkyl.

Suitably R⁵ is hydrogen or C₁₋₄alkyl.

Suitably R⁶ is hydrogen or C₁₋₄alkyl.

Suitably R⁷ is hydrogen or C₁₋₄alkyl.

Suitably R⁸ represents hydrogen, CF₃, or C₁₋₃alkyl. In one aspect R⁸ is hydrogen, CH₃ or CF₃. In another aspect R⁸ is hydrogen or CF₃. In an alternative aspect R⁸ is CH₃. In a yet further aspect R⁸ is Cl.

Suitably R⁹ is hydrogen.

Suitably Q^{a} and Q^{b} are each hydrogen.

Examples of the compounds of formula (I) include the compounds of Examples 1 to 80 and derivatives thereof.

In one aspect examples of the compounds of formula (I) include the compounds of Examples 1 to 56, 69 to 72, 74, 75, and 78:
6-{2-[5-chloro-2-benzyloxy-phenyl]-5-methyl-pyrrol-1-yl}-picolinic acid;
3-{2-[5-chloro-2-(2,6-difluoro-benzyloxy)-phenyl]-5-methyl-pyrrol-1-yl}-6-methyl-benzoic acid;
3-{2-[5-chloro-2-(2,4-dimethyl-benzyloxy)-phenyl]-5-methyl-pyrrol-1-yl}-6-methyl-benzoic acid;
3-{2-[5-chloro-2-(2,6-dichloro-benzyloxy)-phenyl]-5-methyl-pyrrol-1-yl}-6-methyl-benzoic acid;
3-{2-[5-chloro-2-(3,4-difluoro-benzyloxy)-phenyl]-5-methyl-pyrrol-1-yl}-6-methyl-benzoic acid;
3-{2-[5-chloro-2-(2-fluoro-4-trifluoromethyl-benzyloxy)-phenyl]-5-methyl-pyrrol-1-yl}-6-methyl-benzoic acid;
3-{2-[5-chloro-2-(2-methyl-benzyloxy)-phenyl]-5-methyl-pyrrol-1-yl}-6-methyl-benzoic acid;
3-{2-[5-chloro-2-(4-trifluoromethyl-benyloxy)-phenyl]-5-methyl-pyrrol-1-yl}-6-methyl-benzoic acid;
3-{2-[5-chloro-2-(2,5-difluoro-benzyloxy)-phenyl]-5-methyl-pyrrol-1-yl}-6-methyl-benzoic acid;
3-{2-[5-chloro-2-(2-chloro-benzyloxy)-phenyl]-5-methyl-pyrrol-1-yl}-6-methyl-benzoic acid;
3-{2-[5-chloro-2-(2,3,6-trifluoro-benzyloxy)-phenyl]-5-methyl-pyrrol-1-yl}-6-methyl-benzoic acid;
3-{2-[5-chloro-2-(2-chloro-6-fluoro-benzyloxy)-phenyl]-5-methyl-pyrrol-1-yl}-6-methyl-benzoic acid;
3-{2-[5-chloro-2-(2-fluoro-4-chloro-benzyloxy)-phenyl]-5-methyl-pyrrol-1-yl}-6-methyl-benzoic acid;
3-{2-[5-chloro-2-(2-chloro-4-fluoro-benzyloxy)-phenyl]-5-methyl-pyrrol-1-yl}-6-methyl-benzoic acid;
3-{2-[5-chloro-2-(2-fluoro-4-bromo-benzyloxy)-phenyl]-5-methyl-pyrrol-1-yl}-6-methyl-benzoic acid;
3-{2-[5-chloro-2-(2-fluoro-benzyloxy)-phenyl]-5-methyl-pyrrol-1-yl}-6-methyl-benzoic acid;
3-{2-[5-chloro-2-(4-chloro-benzyloxy)-phenyl]-5-methyl-pyrrol-1-yl}-6-methyl-benzoic acid;
3-{2-[5-chloro-2-(2,4-dichloro-benzyloxy)-phenyl]-5-methyl-pyrrol-1-yl}-6-methyl-benzoic acid;
3-{2-[5-chloro-2-(2,4-difluoro-benzyloxy)-phenyl]-5-methyl-pyrrol-1-yl}-6-methyl-benzoic acid;
4-{2-[5-trifluoromethyl-2-(2,4-difluoro-benzyloxy)-phenyl]-5-methyl-pyrrol-1-yl}-6-methyl-benzoic acid;
6-{2-[5-bromo-2-benzyloxy-phenyl]-5-methyl-pyrrol-1-yl}picolinic acid;
6-{2-[5-bromo-2-(2-fluoro-benzyloxy)-phenyl]-5-methyl-pyrrol-1-yl}-picolinic acid;
6-{2-[5-bromo-2-(2-chloro-6-fluoro-benzyloxy)-phenyl]-5-methyl-pyrrol-1-yl}-picolinic acid;
6-{2-[5-bromo-2-(2,6-difluoro-benyloxy)-phenyl]-5-methyl-pyrrol-1-yl}-picolinic acid;
6-{2-[5-bromo-2-(2-chloro-4-fluoro-benzyloxy)-phenyl]-5-methyl-pyrrol-1-yl}-picolinic acid;
6-{2-[5-bromo-2-(2-fluoro-4-chloro-benzyloxy)-phenyl]-5-methyl-pyrrol-1-yl}-picolinic acid;
6-{2-[5-bromo-2-(2,4-dichloro-benzyloxy)-phenyl]-5-methyl-pyrrol-1-yl}-picolinic acid;
6-{2-[5-bromo-2-(4-chloro-benzyloxy)-phenyl]-5-methyl-pyrrol-1-yl}-picolinic acid;
6-{2-[5-bromo-2-(2-fluoro-4-bromo-benzyloxy)-phenyl]-5-methyl-pyrrol-1-yl}-picolinic acid;
6-{2-[5-bromo-2-(2,4-difluoro-benzyloxy)-phenyl]-5-methyl-pyrrol-1-yl}-picolinic acid;
6-{2-[5-chloro-2-(2-chloro-6-fluoro-benzyloxy)-phenyl]-5-methyl-pyrrol-1-yl}-picolinic acid;
6-{2-[5-chloro-2-(2,5-difluoro-benzyloxy)-phenyl]-5-methyl-pyrrol-1-yl}-picolinic acid;
6-{2-[5-chloro-2-(2-chloro-benzyloxy)-phenyl]-5-methyl-pyrrol-1-yl}-picolinic acid;
6-{2-[5-chloro-2-(2-fluoro-4-chloro-benzyloxy)-phenyl]-5-methyl-pyrrol-1-yl}-picolinic acid;
6-{2-[5-chloro-2-(2-chloro-fluoro-benzyloxy)-phenyl]-5-methyl-pyrrol-1-yl}-picolinic acid;
6-{2-[5-chloro-2-(2,6-difluoro-benzyloxy)-phenyl]-5-methyl-pyrrol-1-yl}picolinic acid;
6-{2-[5-chloro-2-(4-trifluoromethyl-benzyloxy)-phenyl]-5-methyl-pyrrol-1-yl}-picolinic acid;
6-{2-[5-chloro-2-(2,4-dichloro-benzyloxy)-phenyl]-5-methyl-pyrrol-1-yl}-picolinic acid;
6-{2-[5-chloro-2-(2-fluoro-4-bromo-benzyloxy)-phenyl]-5-methyl-pyrrol-1-yl}-picolinic acid;
6-{2-[5-chloro-2-(2,4-difluoro-benzyloxy)-phenyl]-5-methyl-pyrrol-1-yl}-picolinic acid;
6-{2-[5-chloro-2-(4-chloro-benzyloxy)-phenyl]-5-methyl-pyrrol-1-yl}-picolinic acid;
6-{2-[5-chloro-2-(2,3,6-trifluoro-benzyloxy)-phenyl]-5-methyl-pyrrol-1-yl}-picolinic acid;
6-{2-[5-chloro-2-(2-fluoro-benzyloxy)-phenyl]-5-methyl-pyrrol-1-yl}-picolinic acid;
6-{2-[5-trifluoromethyl-2-(2-fluoro-4-chloro-benzyloxy)-phenyl]-5-methyl-pyrrol-1-yl}-picolinic acid;
6-{2-[5-trifluoromethyl-2-(2-chloro-benzyloxy)-phenyl]-5-methyl-pyrrol-1-yl}-picolinic acid;
6-{2-[5-trifluoromethyl-2-(2,3,6-trifluoro-benzyloxy)-phenyl]-5-methyl-pyrrol-1-yl}-picolinic acid;
6-{2-[5-trifluoromethyl-2-(2-methyl-benzyloxy)-phenyl]-5-methyl-pyrrol-1-yl}-picolinic acid;
6-{2-[5-trifluoromethyl-2-(2-chloro-6-fluoro-benzyloxy)-phenyl]-5-methyl-pyrrol-1-yl}-picolinic acid;
6-{2-[5-trifluoromethyl-2-(2-fluoro-4-bromo-benzyloxy)-phenyl]-5-methyl-pyrrol-1-yl}-picolinic acid;
6-{2-[5-trifluoromethyl-2-(2,4-difluoro-benzyloxy)-phenyl]-5-methyl-pyrrol-1-yl}-picolinic acid;
6-{2-[5-trifluoromethyl-2-(2-fluoro-benzyloxy)-phenyl]-5-methyl-pyrrol-1-yl}-picolinic acid;
6-{2-[5-trifluoromethyl-2-(2,4,6-trifuoro-benyloxy)-phenyl]-5-methyl-pyrrol-1-yl}-picolinic acid;
6-{2-[5-trifluoromethyl-2-(4-chloro-benzyloxy)-phenyl]-5-methyl-pyrrol-1-yl}-picolinic acid;
6-{2-[5-trifluoromethyl-2-(2-chloro4fluoro-benyloxy)-phenyl]-5-methyl-pyrrol-1-yl}-picolinic acid;
6-{2-[5-trifluoromethyl-2-(2,4-dichloro-benzyloxy)-phenyl]-5-methyl-pyrrol-1-yl}-picolinic acid;
3-{2-[5-chloro-2-(4-fluoro-benzyloxy)-phenyl]-5-methyl-pyrrol-1-yl}-isoquinoline-1-carboxylic acid;
3-{2-[5-chloro-2-(2,4-difluoro-benzyloxy)-phenyl]-5-trifluoromethyl-pyrrol-1-yl}-benzoic acid;
3-{2-[5-chloro-2-(4-fluoro-benzyloxy)-phenyl]-5-trifluoromethyl-pyrrol-1-yl}-benzoic acid;
3-{2-[5-chloro-2-(2,6-difluoro-benzyloxy)-pheny]-5-trifluoromethyl-pyrrol-1}-yl-benzoic acid;
3-{2-[5-chloro-2-(2-fluoro-benzyloxy)-phenyl]-5-trifluoromethyl-pyrrol-1-yl}-benzoic acid;
3-[2-(2-benzyloxy-phenyl)-pyrrol-1-yl]-benzoic acid;
3-[2-(5-chloro-2-benzyloxy-phenyl)-pyrrol-1-yl]-benzoic acid; and
6-{2-[5-chloro-2-(2,4-difluoro-benyloxy)-phenyl)-pyrrol-1-yl]-picolinic acid; and derivatives thereof.

A further set of examples are the compounds of Examples 57-68, 73, 76, 77, 79 and 80:
3-[2-(5-Chloro-2-isobutyloxy-phenyl)-5-methyl-pyrrol-1-yl]-6-methyl benzoic acid;
3-[2-(5-Chloro-2-isobutyloxy-phenyl)-5-methyl-pyrrol-1-yl]-benzoic acid;
3-[2-(5-Bromo-2-isobutyloxy-phenyl)-5-methyl-pyrrol-1-yl]-benzoic acid;
3-[2-(5-Bromo-2-isobutyloxy-phenyl)-5-methyl-pyrrol-1-yl]-6-methyl-benzoic acid;
3-[2-(5-Chloro-2-cyclopentylmethoxy-phenyl)-5-methyl-pyrrol-1-yl]-benzoic acid;
3-[2-(5-Chloro-2-cyclopentylmethoxy-phenyl)-5-methyl-pyrrol-1-yl]-6-methyl-benzoic acid;
3-[2-(5-Bromo-2-cyclopentylmethoxy-phenyl)-5-methyl-pyrrol-1-yl]-benzoic acid;
3-[2-(5-Bromo-2-cyclopentylmethoxy-phenyl)5-methyl-pyrrol-1-yl]-6-methyl-benzoic acid;
3-[2-(5-Chloro-2-isobutyloxy-phenyl)-5-methyl-pyrrol-1-yl]-6-fluoro benzoic acid;
3-[2-(5-Bromo-2-isobutyloxy-phenyl)-5-methyl-pyrrol-1-yl-]6-fluoro-benzoic acid;
3-[2-(5-Bromo-2-cyclopentylmethoxy-phenyl)-5-methyl-pyrrol-1-yl]-6-fluoro-benzoic acid;
3-[2-(5-Chloro-2-cyclopentylmethoxy-phenyl)-5-methyl-pyrrol-1-yl]-6-fluoro-benzoic acid;
3-[2-(5-Bromo-2-isobutyloxy-phenyl)-5-methyl-pyrrol-1-yl]-6-methyl benzoic acid;
3-[2-(5-Chloro-2-isobutyoxy-phenyl)-pyrrol-1-yl]-6-fluoro-benzoic acid;
3-[2-(5-Bromo-2-isobutyoxy-phenyl)-pyrrol-1-yl]-6-fluoro-benzoic acid;
3-[2-(5-Chloro-2-benzyloxy-phenyl)-5-chloro-pyrrol-1-yl]-benzoic acid; and
6-[2-(5-Chloro-2-benzyloxy-phenyl)-5chloro-pyrrol-1-yl]-picolinic acid; and derivatives thereof.

Certain examples of compounds of formula (I) include the compounds of Examples 13, 15, 17, 19, 21, 22, 24, 26, 29, 50, 51, 52, and 53 and derivatives thereof, for example the compounds of Examples 24, 29, 51, and 52 and derivatives thereof.

Derivatives of the compounds of formula (I) include pharmaceutically acceptable derivatives.

The invention is described using the following definitions unless otherwise indicated.

The term “pharmaceutically acceptable derivative” means any pharmaceutically acceptable salt, solvate, ester, or solvate of salt or ester of the compounds of formula (I), or any other compound which upon administration to the recipient is capable of providing (directly or indirectly) a compound of formula (I).

It will be appreciated by those skilled in the art that the compounds of formula (I) may be modified to provide pharmaceutically acceptable derivatives thereof at any of the functional groups in the compounds, and that the compounds of formula (I) may be derivatised at more than one position.

It will be appreciated that, for pharmaceutical use, the salts referred to above will be pharmaceutically acceptable salts, but other salts may find use, for example in the preparation of compounds of formula (I) and the pharmaceutically acceptable salts thereof.

Pharmaceutically acceptable salts include those described by Berge, Bighley and Monkhouse, J. Pharm. Sci., 1977, 66, 1-19. The term “pharmaceutically acceptable salts” refers to salts prepared from pharmaceutically acceptable bases including inorganic bases and organic bases. Salts derived from inorganic bases include aluminum, ammonium, calcium, copper, ferric, ferrous, lithium, magnesium, manganic salts, manganous, potassium, sodium, zinc, and the like. A particular salt is the sodium salt. Salts derived from pharmaceutically acceptable organic bases include salts of primary, secondary, and tertiary amines; substituted amines including naturally occurring substituted amines; and cyclic amines. Particular pharmaceutically acceptable organic bases include arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, procaine, purines, theobromine, triethylamine, trimethylamine, tripropyl amine, tromethamine, and the like. Salts may also be formed from basic ion exchange resins, for example polyamine resins. When the compound of the present invention is basic, salts may be prepared from pharmaceutically acceptable acids, including inorganic and organic acids. Such acids include acetic, benzenesulfonic, benzoic, camphorsulfonic, citric, ethanesulfonic, ethanedisulfonic, fumaric, gluconic, glutamic, hydrobromic, hydrochloric, isethionic, lactic, maleic, malic, mandelic, methanesulfonic, mucic, pamoic, pantothenic, phosphoric, propionic, succinic, sulfuric, tartaric, p-toluenesulfonic acid, and the like.

The compounds of formula (I) may be prepared in crystalline or non-crystalline form, and if crystalline, may be optionally hydrated or solvated. This invention includes in its scope stoichiometric hydrates as well as compounds containing variable amounts of water.

Suitable solvates include pharmaceutically acceptable solvates, such as hydrates.

Solvates include stoichiometric solvates and non-stoichiometric solvates.

The terms “halogen or halo” are used to represent fluorine, chlorine, bromine or iodine, more preferably fluorine, chlorine and bromine.

The term “alkyl” as a group or part of a group means a straight, branched or cyclic chain alkyl group or combinations thereof. Unless hereinbefore defined, examples of alkyl include C₁₋₈alkyl, for example methyl, ethyl, n-propyl, iso-propyl, n-butyl, sec-butyl, iso-butyl, tert-butyl, pentyl, hexyl, 1,1-dimethylethyl, cyclopentyl or cyclohexyl or combinations thereof.

The term “alkoxy” as a group or as part of a group means a straight, branched or cyclic chain alkyl group having an oxygen atom attached to the chain. Unless hereinbefore defined examples of alkoxy include C₁₋₈alkoxy, for example methoxy, ethoxy, n-propoxy, iso-propoxy, n-butoxy, sec-butoxy, iso-butoxy, tert-butoxy, pentoxy, hexyloxy, cyclopentoxy or cyclohexyloxy.

The term “alkenyl” means linear or branched structures and combinations thereof, of the indicated number of carbon atoms, having at least one carbon-to-carbon double bond, wherein hydrogen may be replaced by an additional carbon to carbon double bond. C₃₋₈alkenyl, for example, includes ethenyl, propenyl, 1-methylethenyl, butenyl and the like.

The term “alkynyl” means linear or branched structures and combinations thereof, of the indicated number of carbon atoms, having at least one carbon-to-arbon triple bond. C₃₋₈alkynyl, for example, includes ethynyl, propynyl, butynyl and the like.

The term “heterocyclyl” as a group or as part of a group means an aromatic or non-aromatic five or six membered ring which contains from 1 to 4 heteroatoms selected from nitrogen, oxygen or sulfur and unsubstituted or substituted by, for example, up to three substituents, preferably one or two substituents. Examples of 5-membered heterocyclyl groups include furyl, dioxalanyl, pyrrolyl, oxazolyl, thiazolyl, imidazolyl, oxadiazolyl, thiadiazolyl, triazolyl, triazinyl, isothiazolyl, isoxazolyl, thiophenyl, pyrazolyl or tetrazolyl. Examples of 6-membered heterocyclyl groups are pyridyl, pyridazinyl, pyrimidinyl, pyrazinyl, triazinyl or tetrazinyl.

The term “bicyclic heterocyclyl” when used herein means a fused bicyclic aromatic or non-aromatic bicyclic heterocyclyl ring system comprising up to four, preferably one or two, heteroatoms each selected from oxygen, nitrogen and sulphur. Each ring may have from 4 to 7, preferably 5 or 6, ring atoms. A bicyclic heteroaromatic ring system may include a carbocyclic ring. Examples of bicyclic heterocyclyl groups include quinolinyl, isoquinolinyl, quinoxalinyl, quinazolinyl, pyridopyrazinyl, benzoxazolyl, benzothiophenyl, benzimidazolyl, benzothiazolyl, benzoxadiazolyl, benzthiadiazolyl, indolyl, benztriazolyl or naphthyridinyl. Suitably the bicyclic heterocyclyl group is isoquinolinyl.

The term “aryl” as a group or as part of a group means a 5- or 6- membered aromatic ring for example phenyl, or a 7 to 12 membered bicyclic ring system where at least one of the rings is aromatic, for example naphthyl. An aryl group may be substituted by up to four, preferably one to three substitutes. In one aspect the aryl group is phenyl.

The term “heteroaryl” as a group or as part of a group means a monocyclic five or six membered aromatic ring, or a fused bicyclic aromatic ring system comprising two of such monocyclic five or six membered aromatic rings. These heteroaryl rings contain one or more heteroatoms selected from nitrogen, oxygen or sulfur, where N-oxides, sulfur oxides and sulfur dioxides are permissible heteroatom substitutions. A heteroaryl group may be optionally substituted by one or more substitutes for example one or two substitutes. Examples of “heteroaryl” used herein include furyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyrimidinyl, quinolinyl, isoquinolinyl, benzofuryl, benzothienyl, indolyl, and indazolyl.

Optional substituents for alkyl, alkenyl or alkynyl groups unless hereinbefore defined include OH, CO₂R⁴, NR⁴R⁵, (O), —OC₁₋₆alkyl or halo e.g. Cl, Br or F, wherein R⁴, and R⁵ are as hereinbefore defined for compounds of formula (I). An alkyl or alkenyl group may be substituted by one or more optional substituents, for example up to 5, 4, 3, or 2 optional substituents. Particular substituted alkyl groups include those subsituted by one or more fluorine atoms e.g. CF₃.

Optional substituents for alkoxy groups unless herminbefore defined include OH, and halo e.g. Cl, Br or F. An alkoxy group may be substituted by one or more optional substituents, for example up to 5, 4, 3, or 2 optional substituents.

Unless otherwise defined, certain optional substituents for A, aryl, heteroaryl or heterocyclyl groups include halogen, C₁₋₆alkyl, and C₁₋₆alkoxy.

When the heteroatom nitrogen replaces a carbon atom in a C₁₋₈alkyl group, or when nitrogen is present in a heteroaryl, heterocyclyl or bicyclic heterocyclyl group the nitrogen atom will, where appropriate be substituted by one or two substituents selected from hydrogen and C₁₋₈alkyl, preferably hydrogen and C₁₋₆alkyl, more preferably hydrogen.

Compounds of formula (I) wherein can be prepared as set forth in the following scheme and in the examples. The following processes form another aspect of the present invention. 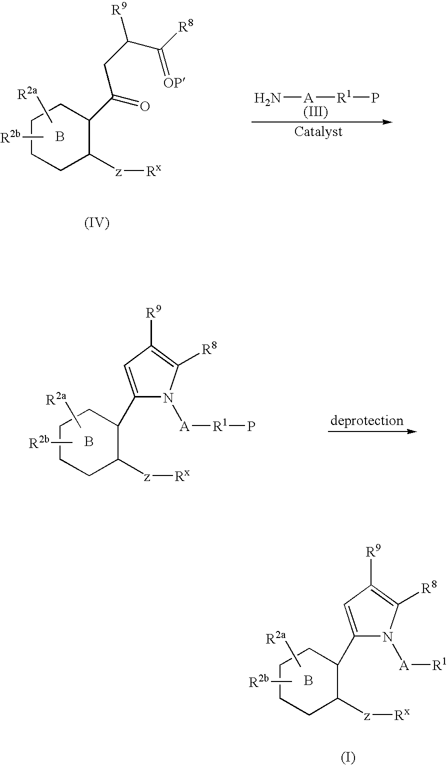
wherein P is an optional protecting group for example methyl or ethyl esters; ═OP′ represents an optional carbonyl protecting group; A, B, Z, R^{2a}, R^{2b}, R¹ and R^{x} are as defined for compounds of formula (I), R⁸ is hydrogen, C₁₋₃alkyl or CF₃ and R⁹ is hydrogen, C₁₋₃alkyl or CF₃.

Compounds of formula (I) wherein R⁸ is Cl may be prepared by treatment of a compound of formula (I) wherein R⁸ is hydrogen with a suitable chlorinating reagent, for example N-chlorosuccinimide. This is most conveniently carried out prior to the deprotection step as illustrated in the Examples.

Similarly, compounds of formula (I) wherein R⁹ is halogen may be prepared by treating a compound of formula (I) wherein R⁹ is hydrogen with the appropriate N-halosuccinimide, for example N-chlorosuccinimide, N-bromosuccinimide or N-iodosuccinimide.

The skilled person will recognise when the use of a protecting group is necessary. When R¹ is CO₂H, a suitable protecting group P is an ester forming group such as C₁₋₄alkyl or optionally substituted benzyl. Suitable reaction conditions for the deprotection of a compound of formula (II) include hydrolysis effected by e.g. heating in ethanolic sodium hydroxide solution, or hydrogenation. Suitable carbonyl protecting groups include acetals and cyclic acetals, e.g. 1,3-dioxolane.

Suitable reaction conditions for the reaction of a compound of formula (IV) with a compound of formula (III) to give a compound of formula (II) include heating with p-toluenesulfonic acid catalyst in toluene solution or heating with with p-toluenesulfonic acid catalyst in N-methyl pyrrolidinone solution. Heating may be effected using microwave radiation.

Accordingly the present invention also provides a process for the preparation of a compound of formula (I) or a derivative thereof: 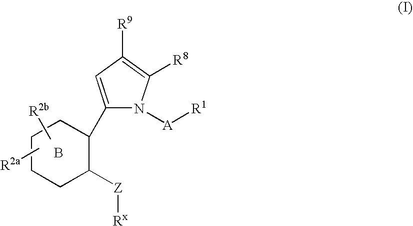
wherein:
A represents an optionally substituted aryl, or an optionally substituted 5- or 6- membered heterocyclyl ring, or an optionally substituted bicyclic heterocyclyl group;
B represents a phenyl or pyridyl ring;
Z represents O, S, SO, or SO₂;
R¹ represents CO₂R⁴, CN, CONR⁵R⁶, CH₂CO₂R⁴, OR⁴, optionally substituted alkyl, optionally substituted alkenyl, optionally substituted SO₂alkyl, SO₂NR⁵R⁶, NR⁵CONR⁵R⁶, COalkyl, 2H-tetrazol-5-yl-methyl, optionally substituted bicyclic heterocycle or optionally substituted heterocyclyl;
R^{2a} and R^{2b} each independently represent hydrogen, halogen, optionally substituted alkyl, optionally substituted alkoxy, CN, SO₂alkyl, SR⁵, NO₂, optionally substituted aryl, CONR⁵R⁶ or optionally substituted heteroaryl;
R^{x} represents optionally substituted alkyl wherein 1 or 2 of the non-terminal carbon atoms are optionally substituted by a group independently selected from NR⁴, O and SOₙ, wherein n is 0, 1 or 2; optionally substituted alkenyl; or optionally substituted alkynyl: or R^{x} represents optionally substituted alkenyl, optionally substituted CQ^{a}Q^{b}-heterocyclyl, optionally substituted CQ^{a}Q^{b}-bicyclic heterocyclyl or optionally substituted CQ^{a}Q^{b}-aryl;R⁴ represents hydrogen or an optionally substituted alkyl;
R⁵ represents hydrogen or an optionally substituted alkyl;
R⁶ represents hydrogen or optionally substituted alkyl, optionally substituted heteroaryl, optionally substituted SO₂aryl, optionally substituted SO₂alkyl, optionally substituted SO₂heteroaryl, CN, optionally substituted CQ^{a}Q^{b}aryl, optionally substituted CQ^{a}Q^{b}heteroaryl or COR⁷;
R⁷ represents hydrogen, optionally substituted alkyl, optionally substituted heteroaryl or optionally substituted aryl;
R⁸ represents hydrogen, CF₃, Cl or C₁₋₃alkyl;
R⁹ represents halogen, hydrogen, CF₃, or C₁₋₃alkyl;
Q^{a} and Q^{b} are each independently selected from hydrogen and CH₃;
wherein when A is a 6-membered ring the R¹ substituent and pyrrole ring are attached to carbon atoms 1,2-, 1,3- or 1,4- relative to each other, and when A is a five-membered ring or bicyclic heterocyclyl group the R¹ substituent and phenyl ring are attached to substitutable carbon atoms 1,2- or 1,3- relative to each other;
comprising:
reacting a compound of formula (IV): 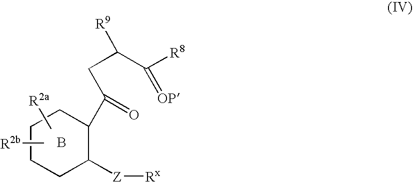
   wherein P′ represents an optional carbonyl protecting group, R^{2a}, R^{2b}, R⁸, R⁹, B, Z and R^{x} are as hereinbefore defined above for a compound of formula (I); with a compound of formula (III):
   H₂N—A—R¹—P (III)
   wherein A and R¹ are as hereinbefore defined above for a compound of formula (I) and P is an optional protecting group;
and where required, and in any order,
converting:
one group R⁸ to another group R⁸; and/or
one group R⁹ to another group R⁹, and/or
one group R^{x} to another group R^{x}; and/or
one group R¹ to another group R¹; and/or
effecting deprotection; and/or
forming a derivative of the compound of formula (I) so formed.

It will be appreciated that certain substituents in intermediates and compounds of formula (I) may be converted to other substituents by conventional methods known to those skilled in the art.

A group R¹ may be converted to another group R¹ by use of conventional organic transformations known to those skilled in the art. For example R¹ ═CO₂H may be converted to an amide, e.g. CONHCQ^{a}Q^{b} aryl or CONHCQ^{a}Q^{b}heteroaryl wherein Q^{a} and Q^{b} are selected from hydrogen and CH₃, by conventional methods for the preparation of amides as described in, for example, Richard Larock, Comprehensive Organic Transformations, 2nd edition, Wiley-VCH, ISBN 0-471-19031-b 4.

Certain substituents in any of the reaction intermediates and compounds of formula (I) may be converted to other substituents by conventional methods known to those skilled in the art. Examples of substituents which may be converted include one group R^{x} to another group R^{x}; and one substituent on a group A to another substituent on a group A. Examples of such transformations include the reduction of a nitro group to give an amino group; alkylation and amidation of amino groups; hydrolysis of esters, alkylation of hydroxy and amino groups; and amidation and esterification of carboxylic acids. Such transformations are well known to those skilled in the art and are described in for example, Richard Larock, Comprehensive Organic Transformations, 2nd edition, Wiley-VCH, ISBN 0-471-19031-4.

For example, when R^{x} is p-methoxybenzyl, cleavage of the ether to give the phenol or pyridinol is carried out using, for example, using acid e.g. HCl/dioxane or HBr/acetic acid or 2,3-dichloro-5,6-dicyano-1,4-benzoquinone (DDQ). When R^{x} is methyl, cleavage of the ether to give the phenol is carried out using, for example, sodium methanethiolate. Cleavage of the ether to give a pyridinol is carried out in the presence of, for example, trifluoroacetic acid. Conversion to another R^{x} group, for example a substituted benzyl group, may be effected by reaction of the phenol or pyridinol with a suitable substituted benzyl bromide. The skilled person will appreciate that conversion of the protecting group P to another protecting group P may also occur under the reaction conditions used. When R^{x} is benzyl, cleavage of the ether to give the phenol or pyridinol may be carried out by hydrogenation according to known methods e.g. H₂—Pd/C or NH₄CO₂H—Pd/C. The resulting phenol or pyridinol can then be converted to another group R^{x} as described above.

It will be appreciated by those skilled in the art that it may be necessary to protect certain reactive substituents during some of the above procedures. The skilled person will recognise when a protecting group is required. Standard protection and deprotection techniques, such as those described in Greene T. W. ‘Protective groups in organic synthesis’, New York, Wiley (1981), can be used. For example, carboxylic acid groups can be protected as esters. Suitably a carbonyl group may be protected as an acetal or cyclic acetal group. Deprotection of such groups is achieved using conventional procedures known in the art. It will be appreciated that protecting groups may be interconverted by conventional means.

Compounds of formula (IV) when R⁸ is other than hydrogen (compounds of formula (IV′)) may be prepared via the following route: 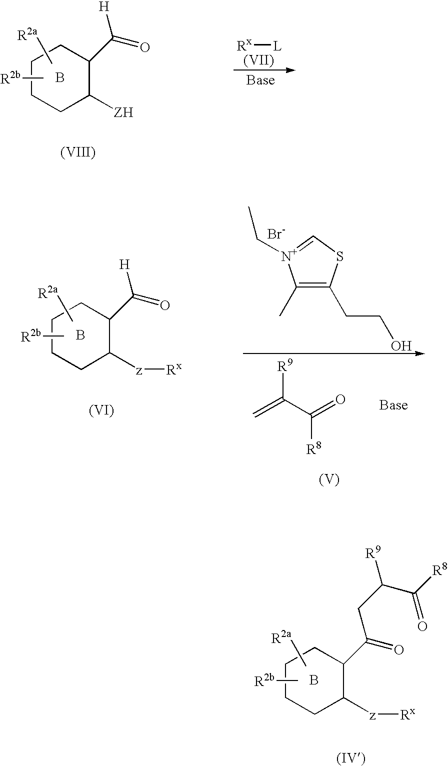
wherein L is a leaving group for example halo, e.g. bromo, or tosylate; B, Z, R^{2a}, R^{2b}, and R^{x} are as defined for compounds of formula (I), R⁸ is CF₃or C₁₋₃alkyl, and R⁹ is hydrogen, C₁₋₆alkyl or CF₃.

Suitable reaction conditions for the conversion of a compound of formula (VI) to a compound of formula (IV′) include heating the compound of formula (VI) with a vinyl ketone of formula (V) in the presence of a thiazolium salt e.g. 3-ethyl-5-(2-hydroxyethyl)-4-methylthiazolium bromide and an organic base, for example triethylamine, in a solvent, for example ethanol.

Suitable reaction conditions for the preparation of a compound of formula (VI) include reacting a salicylaldehyde of formula (VII) with a compound R^{x}—L of formula (VII) in a solvent such as N,N-dimethylformamide, 2-butanone, acetone or tetrahydrofuran in the presence of base, e.g. potassium carbonate.

Compounds of formula (IV) when R⁸ is hydrogen may be prepared for example via the following route: 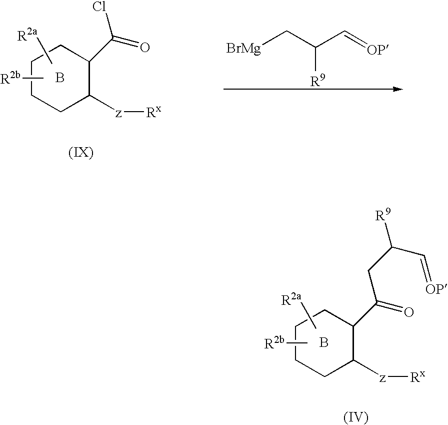
wherein ═OP′ represents a protected carbonyl group, for example an acetal or cyclic acetal group e.g. a 1,3-dioxolane group; B, Z, R^{2a}, R^{2b}, and R^{x} are as defined for compounds of formula (I), and R⁹ is hydrogen, C₁₋₆alkyl or CF₃.

Compounds of formula (III), (V), (VII), (VIII), (IX) and 3-ethyl-5-(2-hydroxyethyl)-4-methylthiazolium bromide are commercially available, or readily prepared by methods known to those skilled in the art.

Compounds of formula (III):
H₂N—A—R¹—P (III)
wherein P is an optional protecting group and R¹ and A are as hereinbefore defined for compounds of formula (I), are commercially available or may readily be prepared from commercially available materials according to known methods for preparing amines, e.g. using methods as described in the Examples. Methods for the preparation of amines are reviewed in The Amino Group, S. Patai (Ed), Interscience, New York 1968, and references cited therein. The preparation of amines is also described in Richard Larock, Comprehensive Organic Transformations, 2nd edition, pages 753 to 879, Wiley-VCH, ISBN 0-471-19031-4.

Intermediates of formula (V): 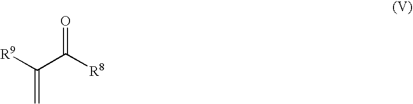
wherein R⁸and R⁹ are as hereinbefore defined for compounds of formula (I) are commercially available or may be readily prepared according to known methods for the preparation of vinyl ketones. For example, F₃CCOCHCH₂═CH₂ may be prepared according to the method of M. Tordeux et al, J. Fluorine Chemistry, 1982, 20(3), 301-306.

Intermediates of formula (VII):
R^{x}—L (VII)
wherein L is as defined above and R^{x} is as defined for compounds of formula (I) are commercially available, or may be readily prepared by known transformations of commercially available compounds.

Intermediates of formula (VIII): 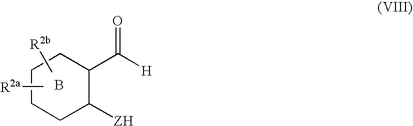
wherein R^{2a}, R^{2b}, Z and B are as defined for compounds of formula (I) are commercially available, or may readily be prepared by methods known to those skilled in the art, for example from suitable commercially available starting materials using methods as described in the examples. The preparation of aldehydes is reviewed in The Chemistry of the Carbonyl Group, S. Patai (Ed), Interscience, New York, 1966, and references cited therein.

Intermediates of formula (IX): 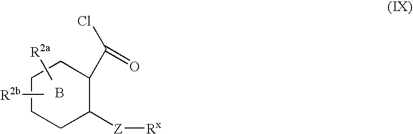
wherein R^{2a}, R^{2b}, Z and B are as defined for compounds of formula (I) are commercially available, or may readily be prepared by methods known to those skilled in the art, for example from suitable commercially available starting materials using methods as described in the examples. The preparation of acid chlorides is reviewed in The Chemistry of the Carbonyl Group, S. Patai (Ed), Interscience, New York, 1966, and references cited therein.

It is to be understood that the present invention encompasses all isomers of formula (I) and their pharmaceutically acceptable derivatives, including all geometric, tautomeric and optical forms, and mixtures thereof (e.g. racemic mixtures). Where additional chiral centres are present in compounds of formula (I), the present invention includes within its scope all possible diastereoismers, including mixtures thereof. The different isomeric forms may be separated or resolved one from the other by conventional methods, or any given isomer may be obtained by conventional synthetic methods or by stereospecific or asymmetric syntheses.

The compounds of the invention bind to the EP₁ receptor and they are therefore considered to be useful in treating conditions mediated by the action of PGE₂ at EP₁ receptors.

Conditions mediated by the action of PGE₂ at EP₁ receptors include pain; fever; inflammation; immunological diseases; abnormal platelet function diseases; impotence or erectile dysfunction; bone disease; hemodynamic side effects of non-steroidal anti-inflammatory drugs; cardiovascular diseases; neurodegenerative diseases and neurodegeneration; neurodegeneration following trauma; tinnitus; dependence on a dependence-inducing agent; complications of Type I diabetes; and kidney dysfunction.

The compounds of formula (I) are considered to be useful as analgesics. They are therefore considered useful in the treatment or prevention of pain.

The compounds of formula (I) are considered useful as analgesics to treat acute pain, chronic pain, neuropathic pain, inflammatory pain, visceral pain, pain associated with cancer and fibromyalgia, pain associated with migraine, tension headache and cluster headaches, and pain associated with functional bowel disorders, non-cardiac chest pain and non-ulcer dyspepsia.

The compounds of formula (I) are considered useful in the treatment of chronic articular pain (e.g. rheumatoid arthritis, osteoarthritis, rheumatoid spondylitis, gouty arthritis and juvenile arthritis) including the property of disease modification and joint structure preservation; musculoskeletal pain; lower back and neck pain; sprains and strains; neuropathic pain; sympathetically maintained pain; myositis; pain associated with cancer and fibromyalgia; pain associated with migraine; pain associated with influenza or other viral infections, such as the common cold; rheumatic fever; pain associated with functional bowel disorders such as non-ulcer dyspepsia, non-cardiac chest pain and irritable bowel syndrome; pain associated with myocardial ischemia; post operative pain; headache; toothache; and dysmenorrhea.

The compounds of the invention are considered to be particularly useful in the treatment of neuropathic pain. Neuropathic pain syndromes can develop following neuronal injury and the resulting pain may persist for months or years, even after the original injury has healed. Neuronal injury may occur in the peripheral nerves, dorsal roots, spinal cord or certain regions in the brain. Neuropathic pain syndromes are traditionally classified according to the disease or event that precipitated them. Neuropathic pain syndromes include: diabetic neuropathy; sciatica; non-specific lower back pain; multiple sclerosis pain; fibromyalgia; HIV-related neuropathy; post-herpetic neuralgia; trigeminal neuralgia; and pain resulting from physical trauma, amputation, cancer, toxins or chronic inflammatory conditions. These conditions are difficult to treat and although several drugs are known to have limited efficacy, complete pain control is rarely achieved. The symptoms of neuropathic pain are heterogeneous and are often described as spontaneous shooting and lancinating pain, or ongoing, burning pain. In addition, there is pain associated with normally non-painful sensations such as “pins and needles” (paraesthesias and dysesthesias), increased sensitivity to touch (hyperesthesia), painful sensation following innocuous stimulation (dynamic, static or thermal allodynia), increased sensitivity to noxious stimuli (thermal, cold, mechanical hyperalgesia), continuing pain sensation after removal of the stimulation (hyperpathia) or an absence of or deficit in selective sensory pathways (hypoalgesia).

The compounds of formula (I) are also considered useful in the treatment of fever.

The compounds of formula (I) are also considered useful in the treatment of inflammation, for example in the treatment of skin conditions (e.g. sunburn, burns, eczema, dermatitis, psoriasis); ophthalmic diseases such as glaucoma, retinitis, retinopathies, uveitis and of acute injury to the eye tissue (e.g. conjunctivitis); lung disorders (e.g. asthma, bronchitis, emphysema, allergic rhinitis, respiratory distress syndrome, pigeon fancier's disease, farmer's lung, chronic obstructive pulmonary disease, (COPD); gastrointestinal tract disorders (e.g. aphthous ulcer, Crohn's disease, atopic gastritis, gastritis varialoforme, ulcerative colitis, coeliac disease, regional ileitis, irritable bowel syndrome, inflammatory bowel disease, gastrointestinal reflux disease); organ transplantation; other conditions with an inflammatory component such as vascular disease, migraine, periarteritis nodosa, thyroiditis, aplastic anaemia, Hodgkin's disease, sclerodoma, myaesthenia gravis, multiple sclerosis, sorcoidosis, nephrotic syndrome, Bechet's syndrome, gingivitis, myocardial ischemia, pyrexia, systemic lupus erythematosus, polymyositis, tendinitis, bursitis, and Sjogren's syndrome.

The compounds of formula (I) are also considered useful in the treatment of immunological diseases such as autoimmune diseases, immunological deficiency diseases or organ transplantation. The compounds of formula (I) are also effective in increasing the latency of HIV infection.

The compounds of formula (I) are also considered useful in the treatment of diseases relating to abnormal platelet function (e.g. occlusive vascular diseases).

The compounds of formula (I) are also considered useful for the preparation of a drug with diuretic action.

The compounds of formula (I) are also considered useful in the treatment of impotence or erectile dysfunction.

The compounds of formula (I) are also considered useful in the treatment of bone disease characterised by abnormal bone metabolism or resorbtion such as osteoporosis (especially postmenopausal osteoporosis), hyper-calcemia, hyperparathyroidism, Paget's bone diseases, osteolysis, hypercalcemia of malignancy with or without bone metastases, rheumatoid arthritis, periodontitis, osteoarthritis, ostealgia, osteopenia, cancer cacchexia, calculosis, lithiasis (especially urolithiasis), solid carcinoma, gout and ankylosing spondylitis, tendinitis and bursitis.

The compounds of formula (I) are also considered useful for attenuating the hemodynamic side effects of non-steroidal anti-inflammatory drugs (NSAID's) and cyclooxygenase-2 (COX-2) inhibitors.

The compounds of formula (I) are also considered useful in the treatment of cardiovascular diseases such as hypertension or myocardiac ischemia; functional or organic venous insufficiency; varicose therapy; haemorrhoids; and shock states associated with a marked drop in arterial pressure (e.g. septic shock).

The compounds of formula (I) are also considered useful in the treatment of neurodegenerative diseases and neurodegeneration such as dementia, particularly degenerative dementia (including senile dementia, Alzheimer's disease, Pick's disease, Huntingdon's chorea, Parkinson's disease and Creutzfeldt-Jakob disease, ALS, motor neuron disease); vascular dementia (including multi-infarct dementia); as well as dementia associated with intracranial space occupying lesions; trauma; infections and related conditions (including HIV infection); metabolism; toxins; anoxia and vitamin deficiency; and mild cognitive impairment associated with ageing, particularly Age Associated Memory Impairment.

The compounds of formula (I) are also considered useful in the treatment of neuroprotection and in the treatment of neurodegeneration following trauma such as stroke, cardiac arrest, pulmonary bypass, traumatic brain injury, spinal cord injury or the like.

The compounds of formula (I) are also considered useful in the treatment of tinnitus.

The compounds of formula (I) are also considered useful in preventing or reducing dependence on, or preventing or reducing tolerance or reverse tolerance to, a dependence-inducing agent. Examples of dependence inducing agents include opioids (e.g. morphine), CNS depressants (e.g. ethanol), psychostimulants (e.g. cocaine) and nicotine.

The compounds of formula (I) are also considered useful in the treatment of complications of Type 1 diabetes (e.g. diabetic microangiopathy, diabetic retinopathy, diabetic nephropathy, macular degeneration, glaucoma), nephrotic syndrome, aplastic anaemia, uveitis, Kawasaki disease and sarcoidosis.

The compounds of formula (I) are also considered useful in the treatment of kidney dysfunction (nephritis, particularly mesangial proliferative glomerulonephritis, nephritic syndrome), liver dysfunction (hepatitis, cirrhosis), gastrointestinal dysfunction (diarrhoea) and colon cancer.

The compounds of formula (I) are also useful in the treatment of overactive bladder and urge incontenance.

It is to be understood that reference to treatment includes both treatment of established symptoms and prophylactic treatment, unless explicitly stated otherwise.

According to a further aspect of the invention, we provide a compound of formula (I) or a pharmaceutically acceptable derivative thereof for use in human or veterinary medicine.

According to another aspect of the invention, we provide a compound of formula (I) or a pharmaceutically acceptable derivative thereof for use in the treatment of a condition which is mediated by the action of PGE₂ at EP₁ receptors.

According to a further aspect of the invention, we provide a method of treating a human or animal subject suffering from a condition which is mediated by the action of PGE₂ at EP₁ receptors which comprises administering to said subject an effective amount of a compound of formula (I) or a pharmaceutically acceptable derivative thereof.

According to a further aspect of the invention we provide a method of treating a human or animal subject suffering from a pain, or an inflammatory, immunological, bone, neurodegenerative or renal disorder, which method comprises administering to said subject an effective amount of a compound of formula (I) or a pharmaceutically acceptable derivative thereof.

According to another aspect of the invention, we provide the use of a compound of formula (I) or a pharmaceutically acceptable derivative thereof for the manufacture of a therapeutic agent for the treatment of a condition which is mediated by the action of PGE₂ at EP₁ receptors.

According to another aspect of the invention we provide the use of a compound of formula (I) or a pharmaceutically acceptable derivative thereof for the manufacture of a therapeutic agent for the treatment or prevention of a condition such as a pain, or an inflammatory, immunological, bone, neurodegenerative or renal disorder.

The compounds of formula (I) and their pharmaceutically acceptable derivatives are conveniently administered in the form of pharmaceutical compositions. Such compositions may conveniently be presented for use in conventional manner in admixture with one or more physiologically acceptable carriers or excipients.

Thus, in another aspect of the invention, we provide a pharmaceutical composition comprising a compound of formula (I) or a pharmaceutically acceptable derivative thereof adapted for use in human or veterinary medicine.

The compounds of formula (I) and their pharmaceutically acceptable derivatives may be formulated for administration in any suitable manner. They may, for example, be formulated for topical administration or administration by inhalation or, more preferably, for oral, transdermal or parenteral administration. The pharmaceutical composition may be in a form such that it can effect controlled release of the compounds of formula (I) and their pharmaceutically acceptable derivatives.

For oral administration, the pharmaceutical composition may take the form of, for example, tablets (including sublingual tablets), capsules, powders, solutions, syrups or suspensions prepared by conventional means with acceptable excipients.

For transdermal administration, the pharmaceutical composition may be given in the form of a transdermal patch, such as a transdermal iontophoretic patch.

For parenteral administration, the pharmaceutical composition may be given as an injection or a continuous infusion (e.g. intravenously, intravascularly or subcutaneously). The compositions may take such forms as suspensions, solutions or emulsions in oily or aqueous vehicles and may contain formulatory agents such as suspending, stabilising and/or dispersing agents. For administration by injection these may take the form of a unit dose presentation or as a multidose presentation preferably with an added preservative.

Alternatively for parenteral administration the active ingredient may be in powder form for reconstitution with a suitable vehicle.

The compounds of the invention may also be formulated as a depot preparation. Such long acting formulations may be administered by implantation (for example subcutaneously or intramuscularly) or by intramuscular injection. Thus, for example, the compounds of the invention may be formulated with suitable polymeric or hydrophobic materials (for example as an emulsion in an acceptable oil) or ion exchange resins, or as sparingly soluble derivatives, for example, as a sparingly soluble salt.

The EP₁ receptor compounds for use In the instant invention may be used in combination with other therapeutic agents, for example COX-2 inhibitors, such as celecoxib, deracoxib, rofecoxib, valdecoxib, parecoxib or COX-189; 5-lipoxygenase inhibitors; NSAID's, such as diclofenac, indomethacin, nabumetone or ibuprofen; leukotriene receptor antagonists; DMARD's such as methotrexate; adenosine A1 receptor agonists; sodium channel blockers, such as lamotrigine; NMDA receptor modulators, such as glycine receptor antagonists; gabapentin and related compounds; tricyclic antidepressants such as amitriptyline; neurone stabilising antiepileptic drugs; mono-aminergic uptake inhibitors such as venlafaxine; opioid analgesics; local anaesthetics; 5HT₁ agonists, such as triptans, for example sumatriptan, naratriptan, zolmitriptan, eletriptan, frovatriptan, almotriptan or rizatriptan; nicotinic acetyl choline (nACh) receptor modulators; glutamate receptor modulators, for example modulators of the NR2B ssubtype; EP₄ receptor ligands; EP₂ receptor ligands; EP₃ receptor ligands; EP₄ agonists and EP₂ agonists; EP₄ antagonists; EP₂ antagonists and EP₃ antagonists; cannabanoid receptor ligands; bradykinin receptor ligands and vanilloid receptor ligand. When the compounds are used in combination with other therapeutic agents, the compounds may be administered either sequentially or simultaneously by any convenient route.

Additional COX-2 inhibitors are disclosed in U.S. Pat. No. 5,474,995 U.S. Pat. No. 5,633,272; U.S. Pat. No. 5,466,823, U.S. Pat. No. 6,310,099 and U.S. Pat No. 6,291,523; and in WO 96/25405, WO 97/38986, WO 98/03484, WO 97/14691, WO 99/12930, WO 00/26216, WO 00/52008, WO 00/38311, WO 01/58881 and WO 02/18374.

The invention thus provides, in a further aspect, a combination comprising a compound of formula (I) or a pharmaceutically acceptable derivative thereof together with a further therapeutic agent or agents.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations.

When a compound of formula (I) or a pharmaceutically acceptable derivative thereof is used in combination with a second therapeutic agent active against the same disease state the dose of each compound may differ from that when the compound is used alone. Appropriate doses will be readily appreciated by those skilled in the art.

A proposed daily dosage of compounds of formula (I) or their pharmaceutically acceptable derivatives for the treatment of man is from 0.01 to 30 mg/kg body weight per day and more particularly 0.1 to 10 mg/kg body weight per day, which may be administered as a single or divided dose, for example one to four times per day. The dose range for adult human beings is generally from 8 to 2000 mg/day, such as from 20 to 1000 mg/day, preferably 35 to 200 mg/day.

The precise amount of the compounds of formula (I) administered to a host, particularly a human patient, will be the responsibility of the attendant physician. However, the dose employed will depend on a number of factors including the age and sex of the patient, the precise condition being treated and its severity, and the route of administration.

No unacceptable toxicological effects are expected with compounds of the invention when administered in accordance with the invention.

All publications, including but not limited to patents and patent applications, cited in this specification are herein incorporated by reference as if each individual publication were specifically and individually indicated to be incorporated by reference herein as though fully set forth.

The following non-limiting Examples illustrate the preparation of pharmacologically active compounds of the invention.

### EXAMPLES

Abbreviations:

Bn (benzyl), Bu, Pr, Me, Et (butyl, propyl, methyl ethyl), DMSO (dimethyl sulfoxide), DCM (dichloromethane), DME (ethylene glycol dimethyl ether), DMF (N,N-dimethylformamide), EDC (1-(3-dimethylaminopropyl)3-ethylcarbodiimide), EtOAc (ethyl acetate), EtOH (ethanol), HPLC (High pressure liquid chromatography), LCMS (Liquid chromatography/Mass spectroscopy), MDAP (Mass Directed Auto Preparation), MeOH (methanol), NMR (Nuclear Magnetic Resonance (spectrum)), NMP (n-methyl pyrrolidone), Ph (phenyl), pTSA (para-toluene sulphonic acid), SPE (Solid Phase Extraction), TBAF (tetrabutylammonium fluoride), THF (tetrahydrofuran), s, d, t, q, m, br (singlet, doublet, triplet, quartet, multiplet, broad.)

The term “Biotage” refers to pre-packed silica gel cartridges.

LCMS

Column: 3.3 cm×4.6 mm ID, 3 um ABZ+PLUS
Flow Rate: 3 ml/min
Injection Volume: 5 μl,
Temp: RT

UV Detection Range: 215 to 330 nm

| Solvents: | A: 0.1% Formic Acid + 10 mMolar Ammonium Acetate. | | |
|---|---|---|---|
| | B: 95% Acetonitrile + 0.05% Formic Acid | | |
| | Time | A % | B % |
| Gradient: | 0.00 | 100 | 0 |
| | 0.70 | 100 | 0 |
| | 4.20 | 0 | 100 |
| | 5.30 | 0 | 100 |
| | 5.50 | 100 | 0 |

Mass Directed Autopreparation
Hardware:
Waters 600 gradient pump
Waters 2767 inject/collector
Waters Reagent Manager
Micromass ZMD mass spectrometer
Gilson Aspec—waste collector
Gilson 115 post-fraction UV detector
   Software:
Micromass Masslynx version 4.0
   Column
The column used is typically a Supelco LCABZ++ column whose dimensions are 20 mm internal diameter by 100 mm in length. The stationary phase particle size is 5 μm.
   Solvents:
A: Aqueous solvent=Water+0.1% Formic Acid
B: Organic solvent=MeCN: Water 95:5+0.05% Formic Acid
Make up solvent=MeOH: Water 80:20+50 mMol Ammonium Acetate
Needle rinse solvent=MeOH: Water: DMSO 80:10:10

The method used depends on the analytical retention time of the compound of interest. 15-minute runtime, which comprises a 10-minute gradient followed by a 5-minute column flush and re-equilibration step.
MDP 1.5-2.2=0-30% B
MDP 2.0-2.8=5-30% B
MDP 2.5-3.0=15-55% B
MDP 2.8-4.0=30-80% B
MDP 3.8-5.5=50-90% B
   Flow Rate:
flow rate 20 ml/min.

### 2-Benzyloxy-5-chloro-benzaldehyde

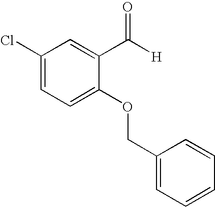

5-Chlorosalicylaldehyde (10.1 g, 64.6 mmol), benzyl bromide (11.5 ml, 96.7 mmol) and K₂CO₃ (17.9 g, 13.0 mmol) were heated in DMF (65 ml) at 60° C. for 18 hrs. Upon cooling to room temperature, Et₂O and H₂O were added. The layers were separated and the aqueous phase was extracted with Et₂O. The combined organic extracts were dried (Na₂SO₄), filtered and concentrated to give the title compound (15.8 g, 100%). ¹H NMR (400 MHz, CDCl₃) 5.18 (2H, s), 7.00 (1H, d, J=9 Hz), 7.32-7.44 (5H, m's excess), 7.47 (1H, dd, J=3 Hz, J=9 Hz), 7.80 (1H, d, J=3 Hz), 10.50 (1H, s).

The following compounds were prepared by a similar route to 2-benzyloxy-5-chloro-benzaldehyde from the appropriate intermediates

| Structure | Name | Data |
|---|---|---|
| 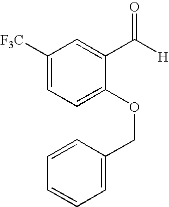 | 2-Benzyloxy-5- trifluoromethyl- benzaldehyde | ¹H-NMR (400 MHz, CDCl₃) 5.27 (2H, s), 7.15 (1H, d, J = 9 Hz), 7.32-7.48 (5H, m), 7.78 (1H, dd, J = 3 Hz, 9 Hz) 8.12 (1H, d, J = 3 Hz) 10.60 (1H, s). LCMS t = 3.59 min |
| 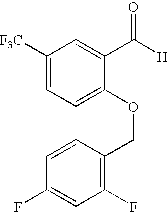 | 2-(2,4-Difluoro- benzyloxy)-5- trifluoromethyl- benzaldehyde | LCMS t = 3.74 |
| 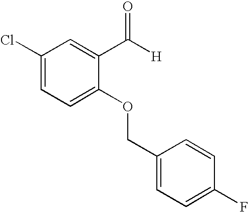 | 5-Chloro-2-(4-fluoro- benzyloxy)-benzaldehyde | LCMS t = 3.56 [MNH₄³⁰ ]282 |
| 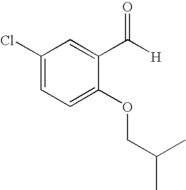 | 5-Chloro-2-isobutoxy- benzaldehyde | LCMS t = 3.63 |
| 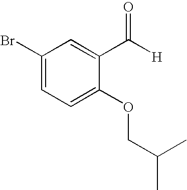 | 5-Bromo-2-isobutoxy- benzaldehyde | LCMS t = 3.70 |

### 5-Chloro-2-cyclopentylmethoxy-benzaldehyde

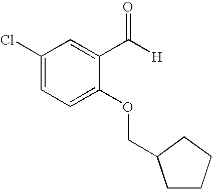

5-Chlorosalicylaldehyde (1 g, 6.4 mmol), cyclopentylmethyl tosylate (1.9 g, 7.5 mmol) and K₂CO₃ (3.5 g, 25.4 mmol) were heated in DMF (10 ml) at 60° C. for 18 hrs. Upon cooling to room temperature, EtOAc and H₂O were added. The layers were separated and the aqueous phase was extracted with EtOAc. The combined organic extracts were dried (MgSO₄), filtered and concentrated. The residue was purified by chromatography using Biotage with isohexane containing a gradient of EtOAc (5-50%) to give the title compound as an oil (0.91 g, 65%).

LCMS t=3.99

The following compounds were prepared by a similar route to 5-chloro-2-cyclopentylmethoxy-benzaldehyde from the appropriate intermediates

| | | |
|---|---|---|
| 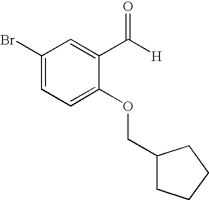 | 5-Bromo-2- cyclopentylmethoxy- benzaldehyde | LCMS t =3.99 |

### 1-(2-Benzyloxy-5-chloro-phenyl)-pentane-1,4-dione

A mixture of 2-benzyloxy-5-chloro-benzaldehyde (4.04 g, 16.41 mmol), methyl vinyl ketone (1.64 ml, 19.7 mmol), 3-ethyl-5-(2-hydroxyethyl)-4-methylthiazolium bromide (654 mg, 2.60 mmol, 0.15 eq) and triethylamine (3.4 ml, 28.7 mmol) were heated in ethanol (5.5 ml, 3M) at 80° C. for 5 hours. Upon cooling, the mixture was diluted with EtOAc and washed with saturated NH₄Cl and saturated NaHCO₃, dried (Na₂SO₄) filtered and concentrated. The residue was purified by chromatography using Biotage with iso-hexane containing a gradient of EtOAc (5-15%) to give the title compound as an oil (4.01 g, 81%).

¹H NMR (400 MHz, CDCl₃) 2.18 (3H, s), 2.78 (2H, d, J=6 Hz), 3.23 (2H, d, J=6 Hz), 5.15 (2H, s), 6.95 (1H, d, J=9 Hz), 7.23-7.50 (6H, m's excess), 7.70 (1H, d, J=3 Hz).

The following compounds were prepared by a similar route to 1-(2-benzyloxy-5-chloro-phenyl)-pentane-1,4-dione from the appropriate intermediates

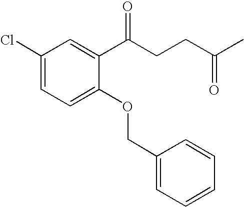

| Structure | Name | Data |
|---|---|---|
| 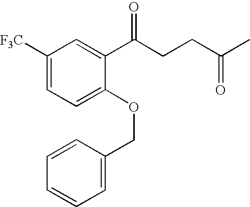 | 1-[5-Trifluoromethyl-2- (benzyloxy)-phenyl]- pentane-1,4-dione | ¹H-NMR (400 MHz, CDCl₃) 2.19 (3H, s), 2.80 (2H, t, J = 6 Hz), 3.26 (2H, t, J = 6 Hz), 5.23 (2H, s), 7.10 (1H, d, J = 9 Hz), 7.35-7.46 (5H, m), 7.68 (1H, dd, J = 3 Hz, 9 Hz), 8.02 (1H, d, J = 3 Hz). LCMS t = 3.51 min |
| 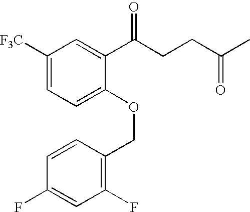 | 1-[5-Trifluoromethyl-2- (2,4-difluorobenzyloxy)- phenyl]-pentane-1,4- dione | LCMS t = 3.62 [MH−] 387 |
| 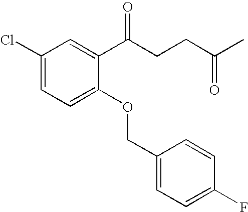 | 1-(5-Chloro-2-(4-fluoro- benzyloxy)-phenyl]- pentane-1,4-dione | LCMS t = 3.46 |
| 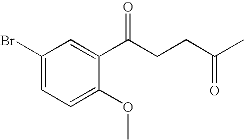 | 1-(5-Bromo-2-methoxy- phenyl)-pentane-1,4- dione | LCMS t = 2.84 min [MH⁺]285/287 |
| 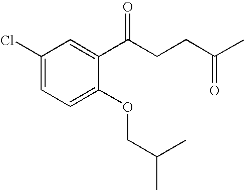 | 1-[5-Chloro-2- isobutyloxyphenyl]- pentane-1,4-dione | LCMS t = 3.48 min [MH⁺]283/285 |
| 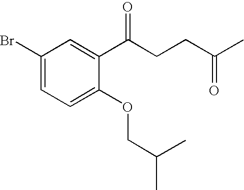 | 1-[5-Bromo-2- isobutytoxyphenyl]- pentane-1,4-dione | LCMS t = 3.54 min [MH⁺]327/329 |
| 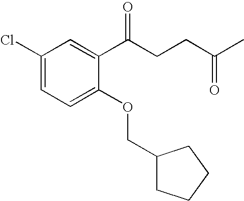 | 1-[5-Chloro-2- cyclopentylmethoxyphenyl]- pentane-1,4-dione | LCMS t = 3.75 min [MNa⁺] 331/333 |
| 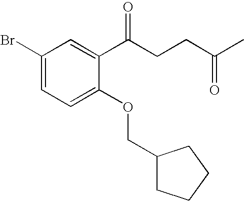 | 1-[5-Bromo-2- cyclopentylmethoxyphenyl]- pentane-1,4-dione | LCMS t = 3.80 min [MH⁺]353/355 |

### 3-[2-(5-Chloro-2-benzyloxy-phenyl)-5-methyl-pyrrol-1-yl]-6-methyl-benzoic acid methyl ester

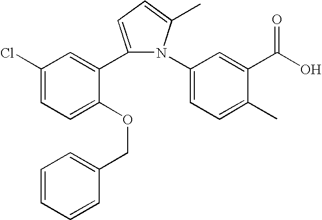

1-[5-Chloro-2-benzyloxy-phenyl]-pentane-1,4-dione (111 mg), 5-amino-2-methyl-benzoic acid (61 mg) and pTSA (cat) were heated in NMP (4.5 mL) at 150° C. for 10 minutes using microwaves. The mixture was diluted with Et₂O washed with 2M HCl, dried (Na₂SO₄), filtered and evaporated. The residue was purified by MDAP, to give the title compound (89 mg).

LCMS t=4.04 min, [MH⁺] 432/434

The following compounds were prepared by a similar route to 3-[2-(5-chloro-2-benzyloxy-phenyl)-5-methyl-pyrrol-1-yl]-6-methyl-benzoic acid methyl ester from the appropriate intermediates

| Structure | Name | Data |
|---|---|---|
| 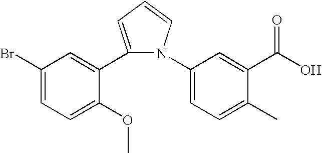 | 3-[2-(5-Bromo-2-methoxy- phenyl)-5-methyl-pyrrol-1- y]-6-methyl-benzoic acid | LCMS t = 3.88 [MH−]398/400 |

### 6-[2-(5-Trifluoromethyl-2-benzyloxy-phenyl)-5-methyl-pyrrol-1-yl]-2-bromo-pyridine

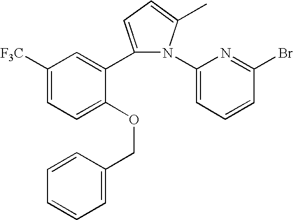

1-(2-Benzyloxy-5-trifluoromethyl-phenyl)-pentane-1,4-dione (1.5 g, 4.3 mmol), 2-amino-6-bromopyridine (0.75 g, 4.3 mmol) and p-TSA (10 mg, cat.) in CH₃CN (5 ml) were heated in a sealed vessel at 200° C. for 1.5 hours using microwaves. Upon cooling the reaction was concentrated and the residue was purified by chromatography using Biotage^{□} with isohexane/EtOAc (5%) as eluant, to give the title compound (645 mg, 31%).

LCMS t=4.14 [MH⁺] 487/489.

The following compounds were prepared by a similar route to 6-[2-(5-trifluoromethyl-2-benzyloxy-phenyl)-5-methyl-pyrrol-1-yl]-2-bromo-pyridine from the appropriate intermediates

| Structure | Name | LCMS |
|---|---|---|
| 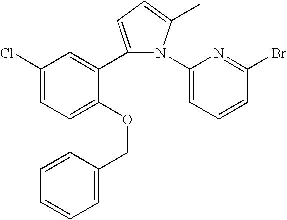 | 6-[2-(5-Chloro-2- benzyloxy-phenyl)-5- methyl-pyrrol-1-yl]-2- bromo-pyridine | t = 4.08 [MH⁺] 453/455 |
| 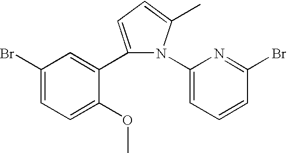 | 6-{2-[5-Bromo-2-methoxy- phenyl]-5-methyl-pyrrol-1- yl}-2-bromo-pyridine | t = 3.93 [MH⁺]421/423/425 |
| 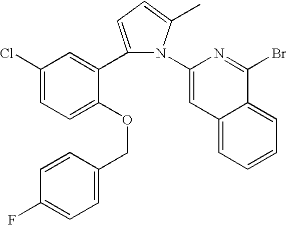 | 1-Bromo-3-{2-(5-chloro-2- (4-fluoro-benzyloxy)- phenyl]-5-methyl-pyrrol-1- yl}-isoquinoline | t = 4.39 [MH⁺]521/523/525 |
| 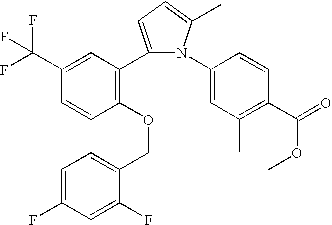 | 4-{2-[5-Trifluoromethyl-2- (2,4-difluoro-benzyloxy)- phenyl]-5-methyl-pyrrol-1- yl}-6-methyl-benzoic acid methyl ester | t = 3.78 [MH^{+] 512} |

### Example 1

### 6-{2-[5-Chloro-2-benzyloxy-phenyl]-5-methyl-pyrrol-1-yl}-picolinic acid

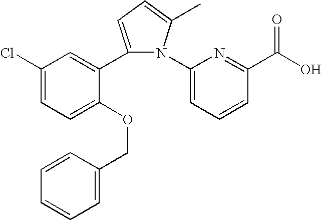

n-Butyl lithium (1.6M in hexanes, 1.44 mL, 2.31 mmol) was added to 6-[2-(5-chloro-2-benzyloxy-phenyl)-5-methyl-pyrrol-1-yl]-2-bromo-pyridine (700 mg, 1.54 mmol) in THF (20 ml) at −78° C. After 1 hour at this temperature, solid CO₂ was added and the solution warmed to room temperature. The reaction mixture was evaporated to dryness, and the resulting yellow oil was triturated with 10% EtOAc/cyclohexane. The mixture was filtered and the residue collected. The filtrate was shown to contain some product, by LCMS, so it was evaporated to dryness, and triturated with 5% EtOAc/cyclohexane, and filtered. The residues were combined. LCMS t=3.81 mins [MH⁺] 419/421.

### 6-[2-(5-Chloro-2-benzyloxy-phenyl)-5-methyl-pyrrol-1-yl]-picolinic acid methyl ester

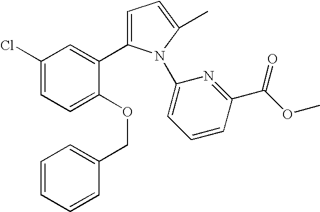

A solution of the acid (400 mg, 0.95 mmol), and thionyl chloride (173 μL, 2.38 mmol) in methanol (5 mL) was stirred at reflux for 3 hours, under N₂. The reaction mixture was evaporated to dryness, and the residue dissolved in CH₂Cl₂. The solution was washed with 2N NaOH(aq). The organics were evaporated to dryness yielding the product was as a brown solid (217 mg, 0.50 mmol, 53%).

¹H NMR (CDCl₃): δ 2.38 (3H, s), 3.94 (3H, s), 4.69 (2H, s), 6.13 (1H, d, J=3 Hz), 6.35 (1H, d, J=3.26 Hz), 6.61 (1H, d J=8 Hz), 7.05-7.09 (3H, m), 7.25-7.30 (5H, m, excess), 7.64 (1H, t, J=7.8 Hz), 7.97 (1H, d, J=7.5 Hz).

The following compound was prepared by a similar route to 6-[2-(5-chloro-2-benzyloxy-phenyl)-5-methyl-pyrrol-1-yl]-picolinic acid methyl ester from the appropriate intermediates

| Structure | Name | LCMS |
|---|---|---|
| 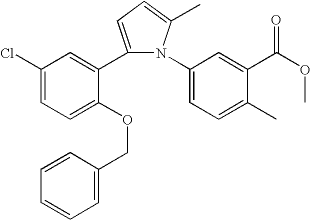 | 3-[2-(5-Chloro-2- benzyloxy-phenyl)-5- methyl-pyrrol-1-yl]-6- methyl-benzoic acid methyl ester | t = 4.13 [MH⁺] 446/448 |

### 3-[2-(5-Chloro-2-hydroxy-phenyl)-5-methyl-pyrrol-1-yl]-6-methyl-benzoic acid methyl ester

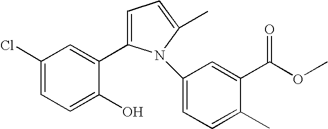

The benzyl ether (2 g, 4.49 mmol) was dissolved in ethanol (50 mL), and the solution was added to Pd(OH)₂ (20% on carbon, 150 mg) under N₂. Hydrogenation was carried out over 75 minutes (130 mL H₂) The reaction mixture was filtered through Celite^{□}, washed through with ethanol, and evaporated to a red oil. The residue was purified by chromatography using a Biotageo® 25S eluting with cyclohexane containing a gradient of 0-3% EtOAc. This yielded the title compound as a yellow oil (846 mg, 2.38 mmol, 53%). LCMS t=3.64 mins [MH⁺] 356/358

### 3-{2-[5-Chloro-2-(2,6-difluoro-benzyloxy)-phenyl]-5-methyl-pyrrol-1-yl}-6-methyl-benzoic acid methyl ester

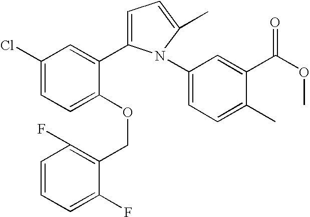

The phenol (141 mg, 0.397 mmol) and the 2,6-difluoro benzyl bromide (82 mg, 0.397 mmol) were heated with potassium carbonate (110 mg, 0.794 mmol) to 60° C. in DMF (1 mL) for 16 hours. The reaction mixture was diluted with H₂O, and extracted twice with Et₂O. The organics were collected, dried over MgSO₄, and evaporated to dryness. The residue was purified by chromatography using an SPE silica cartridge (5 g) eluting with cyclohexane containing a gradient of 0-5% EtOAc. This yielded the title compound as a clear oil (108 mg, 0.225 mmol, 57%). LCMS t=4.08 [MH⁺] 482/484.

The following esters were prepared by a similar route to 3-{2-[5-chloro-2-(2,6-difluoro-benzyloxy)-phenyl]-5-methyl-pyrrol-1 -yl}-6-methyl-benzoic acid methyl ester from the appropriate intermediates.

| Structure | Name | LCMS |
|---|---|---|
| 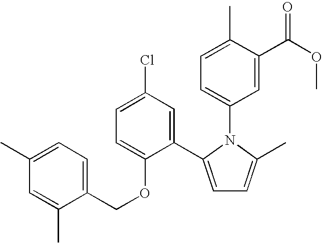 | 3-{2-[5-Chloro-2-(2,4- dimethyl-benzyloxy)- phenyl]-5-methyl-pyrrol- 1-yl}-6-methyl-benzoic acid methyl ester | t = 4.28 [MH⁺] 474/476 |
| 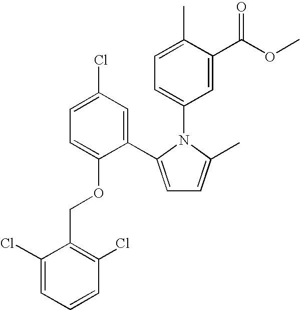 | 3-(2-[5-Chloro-2-(2,6- dichloro-benzyloxy)- phenyl]-5-methyl-pyrrol- 1-yl}-6-methyl-benzoic acid methyl ester | t = 4.24 [MH⁺]514/516/518/520 |
| 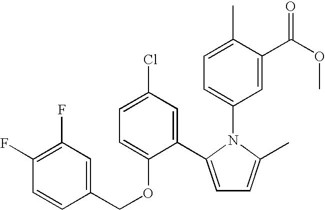 | 3-{2-[5-Chloro-2-(3,4- difluoro-benzyloxy)- phenyl]-5-methyl-pyrrol- 1-yl}-6-methyl-benzoic acid methyl ester | t = 4.17 [MH⁺] 482/484 |
| 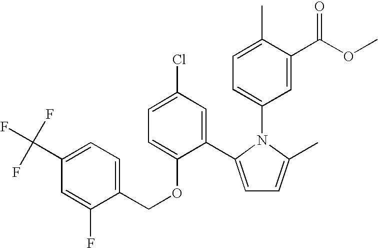 | 3-{2-[5-Chloro-2-(2- fluoro-4-trifluoromethyl- benzyloxy)-phenyl]-5- methyl-pyrrol-1-yl}-6- methyl-benzoic acid methyl ester | t = 4.29 [MH⁺] 532/534 |
| 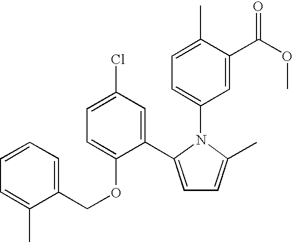 | 3-{2-[5-Chloro-2-(2- methyl-benzyloxy)- phenyl]-5-methyl-pyrrol- 1-yl}-6-methyl-benzoic acid methyl ester | t = 4.22 [MH⁺] 460/462 |
| 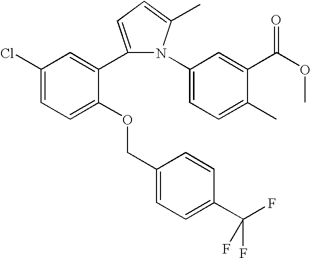 | 3-{2-[5-Chloro-2-(4- trifluoromethyl- benzyloxy)-phenyl]-5- methyl-pyrrol-1-yl}-6- methyl-benzoic acid methyl ester | t = 4.27 [MH⁺] 514/516 |
| 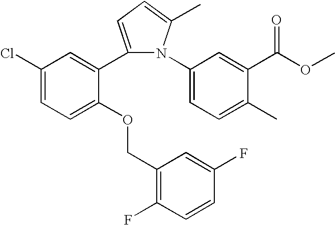 | 3-{2-[5-Chloro-2-(2,5- difluoro-benzyloxy)- phenyl]-5-methyl-pyrrol- 1-yl)-6-methyl-benzoic acid methyl ester | t = 4.30 [MH⁺] 482/484 |
| 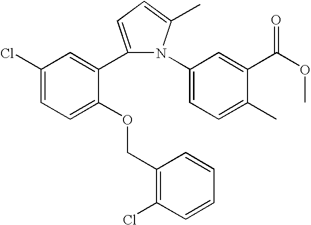 | 3-{2-[5-Chloro-2-(2- chloro-benzyloxy)- phenyl]-5-methyl-pyrrol- 1-yl}-6-methyl-benzoic acid methyl ester | t = 4.41 [MH⁺]480/482/484 |
| 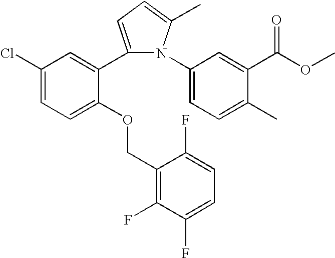 | 3-{2-[5-Chloro-2-(2,3,6- trifluoro-benzyloxy)- phenyl]-5-methyl-pyrrol- 1-yl}-6-methyl-benzoic acid methyl ester | t = 4.23 [MH^{+] 500/502} |
| 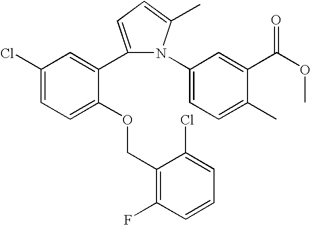 | 3-{2-[5-Chloro-2-(2- chloro-6-fluoro- benzyloxy)-phenyl]-5- methyl-pyrrol-1-yl}-6- methyl-benzoic acid methyl ester | t = 4.31 [MH⁺]498/500/502 |
| 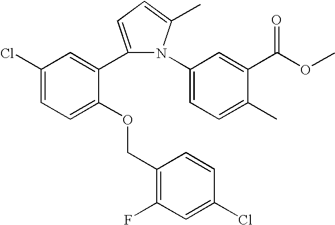 | 3-{2-[5-Chloro-2-(2- fluoro-4-chloro- benzyloxy)-phenyl]-5- methyl-pyrrol-1-yl}-6- methyl-benzoic acid methyl ester | t = 4.30 [MH⁺]498/500/502 |
| 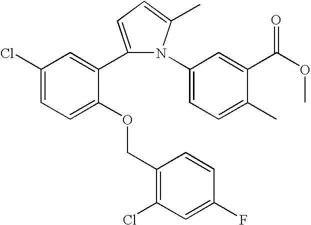 | 3-{2-[5-Chloro-2-(2- chloro-4-fluoro- benzyloxy)-phenyl]-5- methyl-pyrrol-1-yl}-6- methyl-benzoic acid methyl ester | t = 4.31 [MH⁺]498/500/502 |
| 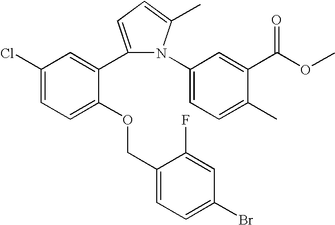 | 3-{2-[5-Chloro-2-(2- fluoro-4-bromo- benzyloxy)-phenyl]-5- methyl-pyrrol-1-yl}-6- methyl-benzoic acid methyl ester | t = 4.32 [MH⁺]542/544/546 |
| 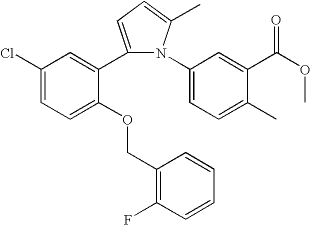 | 3-{2-[5-Chloro-2-(2- fluoro-benzyloxy)- phenyl]-5-methyl-pyrrol- 1-yl}-6-methyl-benzoic acid methyl ester | t = 4.14 [MH⁺] 464/466 |
| 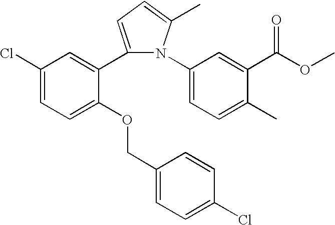 | 3-{2-[5-Chloro-2-(4- chloro-benzyloxy)- phenyl]-5-methyl-pyrrol- 1-yl}-6-methyl-benzoic acid methyl ester | t = 4.24 [MH⁺]480/482/484 |
| 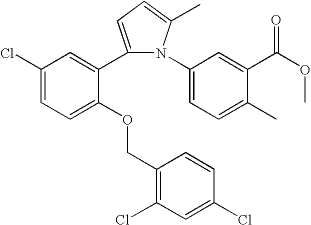 | 3-{2-[5-Chloro-2-(2,4- dichloro-benzyloxy)- phenyl]-5-methyl-pyrrol- 1-yl}-6-methyl-benzoic acid methyl ester | t = 4.41 [MH⁺]514/516/518/520 |
| 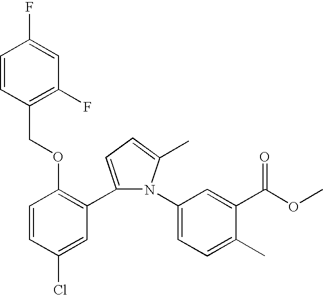 | 3-{2-[5-Chloro-2-(2,4- difluoro-benzyloxy)- phenyl]-5-methyl-pyrrol- 1-yl}-6-methyl-benzoic acid methyl ester | t = 4.14 [MH⁺] 482/484 |

### Example 2

### 3-{2-[5-Chloro-2-(2,6-difluoro-benzyloxy)-phenyl]-5-methyl-pyrrol-1-yl}-6-methyl-benzoic acid

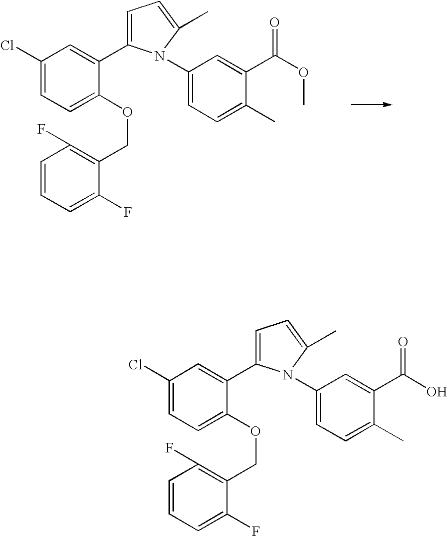

The methyl ester (108 mg, 0.225 mmol) was heated in EtOH (2 mL) with 2N NaOH (0.5 mL, aq) to 120° C. for 5 minutes in the microwave. The reaction mixture was diluted with CH₂Cl₂, shaken with 2N HCl (aq), and separated using a hydrophobic frit. The organics were evaporated to give the title compound as a red gum (64 mg, 0.137 mmol, 61%).

LCMS t=3.89 min [MH⁺] 468/470.

The following acids were prepared by a similar route to 3-{2-[5-chloro-2-(2,6-difluoro-benzyloxy)-phenyl]-5-methyl-pyrrol-1-yl}-6-methyl-benzoic acid from the appropriate intermediates.

| Example | Structure | Name | LCMS |
|---|---|---|---|
| 3 | 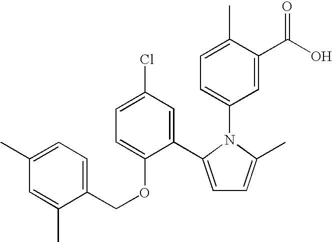 | 3-{2-[5-Chloro-2-(2,4- dimethyl-benzyloxy)- phenyl]-5-methyl-pyrrol- 1-yl}-6-methyl-benzoic acid | t = 4.14 min [MH⁺]460/462 |
| 4 | 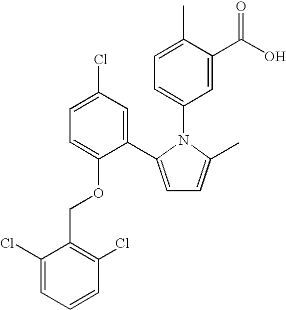 | 3-{2-[5-Chloro-2-(2,6- dichloro-benzyloxy)- phenyl]-5-methyl-pyrrol- 1-yl}-6-methyl-benzoic acid | t = 4.11 min [MH⁺]500/502/504/506 |
| 5 | 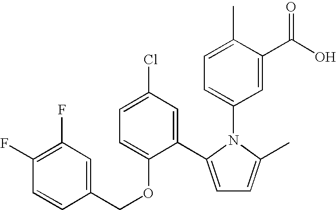 | 3-{2-[5-Chloro-2-(3,4- difluoro-benzyloxy)- phenyl]-5-methyl-pyrrol- 1-yl)-6-methyl-benzoic acid | t = 3.99 min [MH⁺]468/470 |
| 6 | 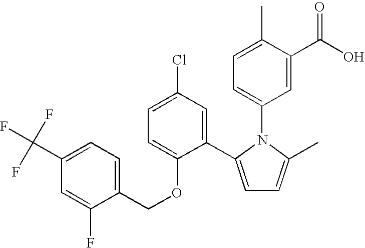 | 3-{2-[5-Chloro-2-(2- fluoro-4-trifluoromethyl- benzyloxy)-phenyl]-5- methyl-pyrrol-1-yl}-6- methyl-benzoic acid | t = 4.11 min [MH⁺]518/520 |
| 7 | 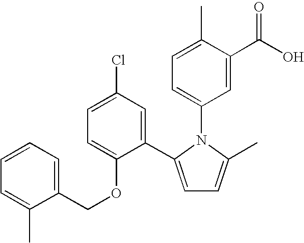 | 3-{2-[5-Chloro-2-(2- methyl-benzyloxy)- phenyl]-5-methyl-pyrrol- 1-yl}-6-methyl-benzoic acid | t = 4.03 min [MH⁺]446/448 |
| 8 | 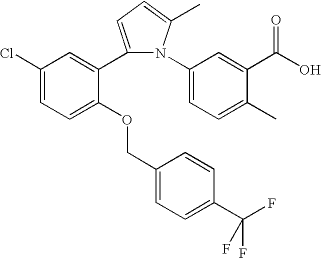 | 3-{2-[5-Chloro-2-(4- trifluoromethyl- benzyloxy)-phenyl]-5- methyl-pyrrol-1-yl}-6- methyl-benzoic acid | t = 4.10 min [MH⁺]500/502 |
| 9 | 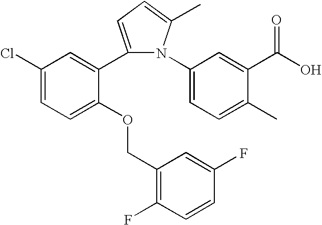 | 3-{2-[5-Chloro-2-(2,5- difluoro-benzyloxy)- phenyl]-5-methyl-pyrrol- 1-yl}-6-methyl-benzoic acid | t = 3.96 min [MH⁺]468/470 |
| 10 | 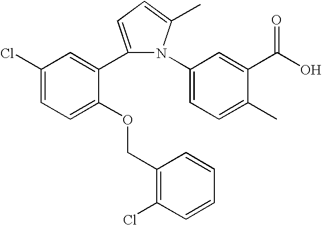 | 3-{2-[5-Chloro-2-(2- chloro-benzyloxy)- phenyl]-5-methyl-pyrrol- 1-yl}-6-methyl-benzoic acid | t = 4.08 min [MH⁺]466/468/470 |
| 11 | 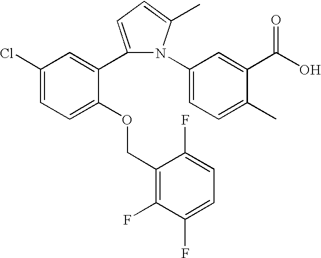 | 3-{2-[5-Chloro-2-(2,3,6- trifluoro-benzyloxy)- phenyl]-5-methyl-pyrrol- 1-yl}-6-methyl-benzoic acid | t = 3.89 min [MH⁺]486/488 |
| 12 | 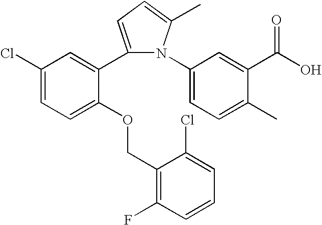 | 3-{2-[5-Chloro-2-(2- chloro-6-fluoro- benzyloxy)-phenyl]-5- methyl-pyrrol-1-yl}-6- methyl-benzoic acid | t = 4.00 min [MH⁺]484/486/488 |
| 13 | 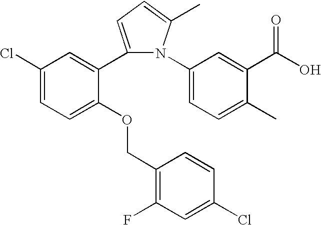 | 3-(2-[5-Chloro-2-(2- fluoro-4-chloro- benzyloxy)-phenyl]-5- methyl-pyrrol-1-yl}-6- methyl-benzoic acid | t = 4.18 min [MH⁺]484/486/488 |
| 14 | 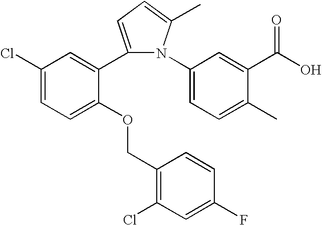 | 3-{2-[5-Chloro-2-(2- chloro-4-fluoro- benzyloxy)-phenyl]-5- methyl-pyrrol-1-yl}-6- methyl-benzoic acid | t = 4.17 min [MH⁺]484/486/488 |
| 15 | 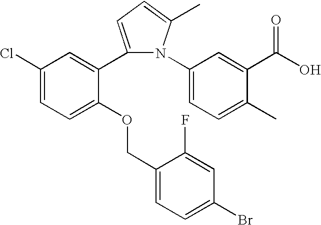 | 3-{2-[5-Chloro-2-(2- fluoro-4-bromo- benzyloxy)-phenyl]-5- methyl-pyrrol-1-yl}-6- methyl-benzoic acid | t = 4.22 min [MH⁺]528/530/532 |
| 16 | 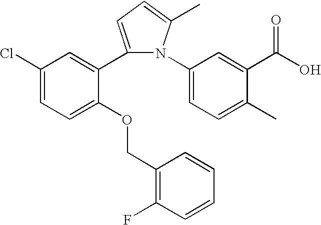 | 3-{2-[5-Chloro-2-(2- fluoro-benzyloxy)- phenyl]-5-methyl-pyrrol- 1-yl}-6-methyl-benzoic acid | t = 4.01 min [MH⁺]450/452 |
| 17 | 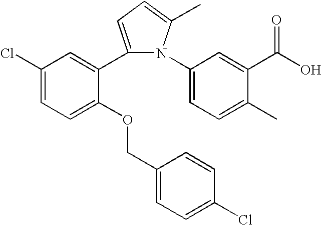 | 3-{2-[5-Chloro-2-(4- chloro-benzyloxy)- phenyl]-5-methyl-pyrrol- 1-yl}-6-methyl-benzoic acid | t = 4.11 min [MH⁺]466/468/470 |
| 18 | 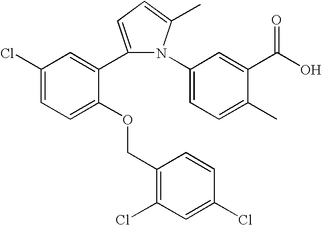 | 3-{2-[5-Chloro-2-(2,4- dichloro-benzyloxy)- phenyl]-5-methyl-pyrrol- 1-yl}-6-methyl-benzoic acid | t = 4.30 min [M⁺]500/502/504/506 |
| 19 | 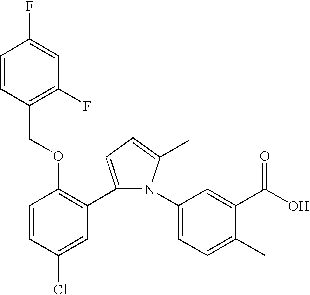 | 3-{2-[5-Chloro-2-(2,4- difluoro-benzyloxy)- phenyl]-5-methyl-pyrrol- 1-yl}-6-methyl-benzoic acid | t = 3.99 min [MH⁺]468/470 |
| 20 | 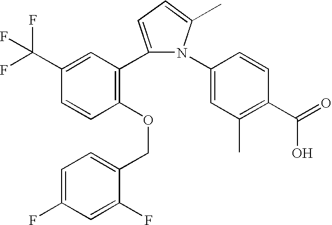 | 4-{2-[5-Trifluoromethyl- 2-(2,4-difluoro- benzyloxy)-phenyl]-5- methyl-pyrrol-1-yl}-6- methyl-benzoic acid | t = 4.04 min [MH⁺]502 |

### 6-[2-(5-Chloro-2-hydroxy-phenyl)-5-methyl-pyrrol-1-yl]-picolinic acid ethyl ester

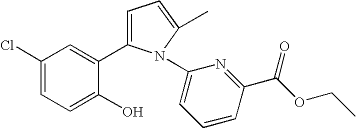

The benzyl ether (300 mg, 0.693 mmol) was dissolved in ethanol (10 mL). The solution was added to palladium hydroxide (100 mg) under N₂. Hydrogenation was carried out over 3 hours (16 mL of H₂ consumed). LCMS showed some remaining starting material. The solution was filtered through Celite, and added to fresh catalyst (100 mg), under N₂. Hydrogenation was continued for 1.5 hours. LCMS showed no remaining starting material. The reaction mixture was filtered through Celite, and evaporated to dryness. ¹H NMR (CDCl₃) showed a mixture of methyl and ethyl esters had formed. The crude product was stirred with EtOH (10 mL) with potassium carbonate (3 eq, 308 mg) at 40° C. to convert all product to ethyl ester form. LCMS confirmed this process was complete. The crude product was purified using column chromatography (SPE, 10 g, Si) eluting with 5% EtOAc/20% CH₂Cl₂/75% cyclohexane, to give the product as brown gum (121 mg, 0.353 mmol, 51%). LCMS t=3.95 mins [MH⁺] 337 and 449

### 6-{2-[5-Bromo-2-methoxy-phenyl]-5-methyl-pyrrol-1-yl}-picolinic acid ethyl ester

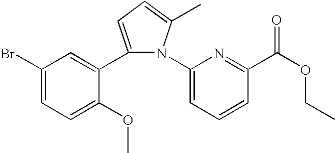

A mixture of 6-{2-[5-bromo-2-methoxy-phenyl]-5-methyl-pyrrol-1-yl}-2-bromo-pyridine (1.15 g, 2.73 mmol), Pd(PPh₃)₂Cl₂ (96 mg, 0.14 mmol), triethylamine (2.25 ml) and ethanol (7.5 ml) were saturated with carbon monoxide gas. The mixture was then heated under reflux, under a carbon monoxide atmosphere for 16 hours. Upon cooling, volatiles were removed in vacuo and the crude oil was purified by chromatography using Biotage® with isohexane/EtOAc (7-25%) as eluant, to give the title compound (710 mg, 63%). LCMS t=3.63 [MH⁺] 415/417.

The following esters were prepared by a similar route to 6-{2-[5-bromo-2-methoxy-phenyl]-5-methyl-pyrrol-1-yl}-picolinic acid ethyl ester from the appropriate intermediates.

| Structure | Name | LCMS |
|---|---|---|
| 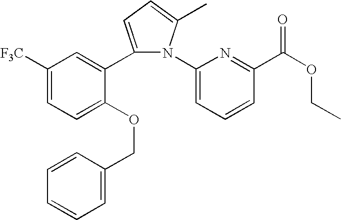 | 6-{2-[5-Trifluoromethyl-2- benzyloxy-phenyl]-5- methyl-pyrrol-1-yl}- picolinic acid ethyl ester | t = 3.99 [MH⁺] 481 |
| 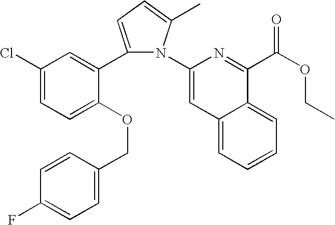 | 3-{2-[5-Chloro-2-(4-fluoro- benzyloxy)-phenyl]-5- methyl-pyrrol-1-yl}- isoquinoline-1-carboxylic acid ethyl ester | t = 4.22 [MH⁺] 515/517 |

### 3-[2-(5-Trifluoromethyl-2-hydroxy-phenyl)-5-methyl-pyrrol-1-yl]-picolinic acid ethyl ester

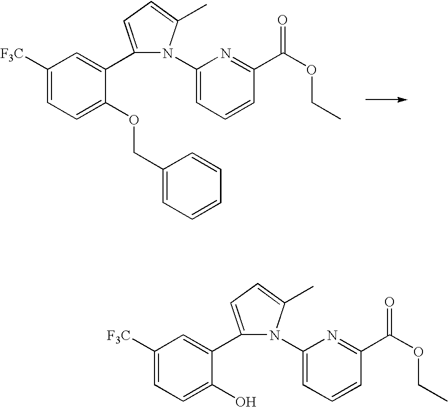

3-[2-(5-Trifluoromethyl-2-benzyloxy-phenyl)-5-methyl-pyrrol-1-yl]-picolinic add ethyl ester (0.32 g, 0.7 mmol), palladium on charcoal (10% containing 50% water) (71 mg), ammonium formate (0.21 g, 3.3 mmol) and EtOH (5 ml) were stirred at 60° C. under a nitrogen atmosphere for 1 hour. Upon cooling the mixture was filtered and the solvent removed in vacuo. The residue was purified by chromatography using a Biotage® 40M eluting with 10% EtOAc/iso-hexane to yield the title compound as a yellow oil (0.226 g, 87%) LCMS t=3.46 [MH⁺] 391

### 6-[2-(5-Bromo-2-hydroxy-phenyl)-5-methyl-pyrrol-1-yl]-picolinic acid

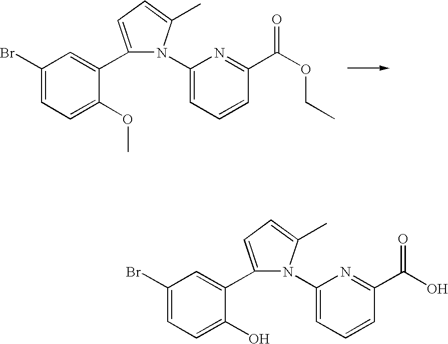

6-{2-[5-Bromo-2-methoxy-phenyl]-5-methyl-pyrrol-1-yl}-picolinic acid ethyl ester (980 mg, 2.36 mmol) and sodium methanethiolate (830 mg, 11.8 mmol) in DMF (75 ml) were heated at 100° C. for 4 hours. Upon cooling, the mixture was diluted with 2M HCl (250 ml) and extracted with EtOAc (3×150 ml). The organics were washed with water (2×50 ml), brine (50 ml), dried (MgSO₄), filtered and concentrated to give the title compound as a yellow oil (1.2 g). LCMS t=3.84 min [MH⁺] 373/375.

The following phenol was prepared by a similar route to 6-[2-(5-bromo-2-hydroxy-phenyl)-5-methyl-pyrrol-1-yl]-picolinic acid from the appropriate intermediates.

| Structure | Name | LCMS |
|---|---|---|
| 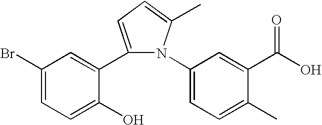 | 3-[2-(5-Bromo-2-hydroxy- phenyl)-5-methyl-pyrrol-1- yl]-6-methyl benzoic acid | t = 3.71 [MH⁺] 384/386 |

### 6-[2-(5-Bromo-2-hydroxy-phenyl)-5-methyl-pyrrol-1-yl]-picolinic acid methyl ester

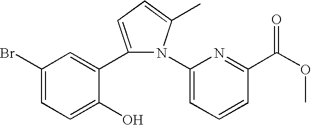

6-[2-(5-Bromo-2-hydroxy-phenyl)-5-methyl-pyrrol-1-yl]-picolinic acid (1 g) and conc. sulfuric acid (0.2 ml) in methanol (10 ml) were heated at 55° C. for 5 hours. Upon cooling, aq NH₃ solution (0.880) (2 ml) was added and the mixture concentrated in vacuo. The residue was partitioned between 2M HCl (50 ml) and EtOAc (50 ml). The organic layer was washed with brine, dried (MgSO₄) filtered and concentrated. The residue was purified by chromatography using Biotage with iso-hexane containing a gradient of EtOAc (10-50%) to give the title compound as an oil (550 mg). LCMS t=3.26 min [MH⁺] 387/389

### 6-{2-[5-Bromo-2-benzyloxy-phenyl]-5-methyl-pyrrol-1-yl}-picolinic acid methyl ester

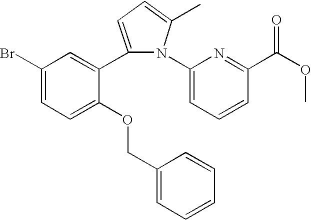

6-[2-(5-Bromo-2-hydroxy-phenyl)-5-methyl-pyrrol-1-yl]-picolinic acid methyl ester (0.11 g, 0.28 mmol), benzyl bromide (0.035 ml, 0.28 mmol) and K₂CO₃ (0.43 g, 0.31 mmol) were heated in butan-2-one (4 ml) under reflux for 16 hours. The mixture was cooled, diluted with CH₂Cl₂ (20 ml) and shaken with water (2 ml). The organics were separated using a phase separator column with a NaSO₄ cartridge attached and concentrated. The residue was purified by chromatography using Biotage®, with iso-hexane/EtOAc (10-15%) as eluant, to give the title compound. LCMS t=3.89 [MH⁺] 477/479.

The following esters were prepared by a similar route to 6-{2-[5-bromo-2-benzyloxy-phenyl]-5-methyl-pyrrol-1-yl}-picolinic acid methyl ester from the appropriate intermediates.

| Structure | Name | LCMS |
|---|---|---|
| 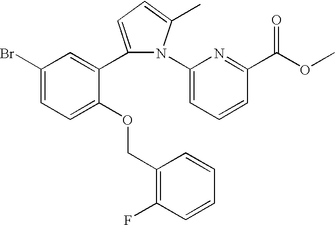 | 6-{2-[5-Bromo-2-(2-fluoro- benzyloxy)-phenyl]-5- methyl-pyrrol-1-yl}- picolinic acid methyl ester | t = 3.91 [MH⁺] 495/497 |
| 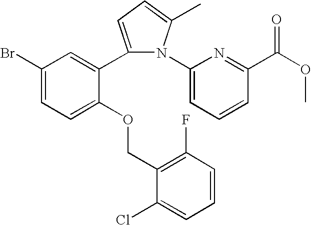 | 6-{2-[5-Bromo-2-(2- chloro-6-fluoro- benzyloxy)-phenyl]-5- methyl-pyrrol-1-yl}- picolinic acid methyl ester | t = 3.96 [MH⁺]529/531/533 |
| 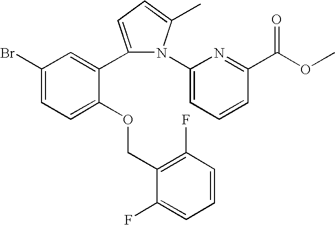 | 6-{2-[5-Bromo-2-(2,6- difluoro-benzyloxy)- phenyl]-5-methyl-pyrrol-1- yl}-picolinic acid methyl ester | t = 3.85 [MH⁺] 513/515 |
| 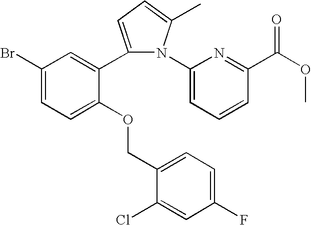 | 6-{2-[5-Bromo-2-(2- chloro-4-fluoro- benzyloxy)-phenyl]-5- methyl-pyrrol-1-yl}- picolinic acid methyl ester | t = 4.12 [MH⁺]529/531/533 |
| 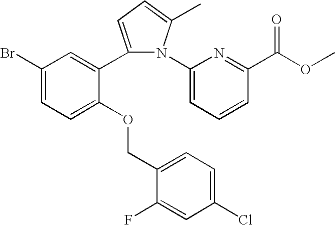 | 6-{2-[5-Bromo-2-(2-fluoro- chloro-benzyloxy)- phenyl]-5-methyl-pyrrol-1- yl}-picolinic acid methyl ester | t = 4.10 [MH⁺]529/531/533 |
| 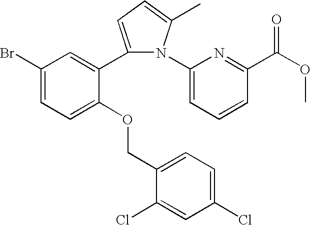 | 6-{2-[5-Bromo-2-(2,4- dichloro-benzyloxy)- phenyl]-5-methyl-pyrrol-1- yl}-picolinic acid methyl ester | t = 4.29 [MH⁺]545/547/549/551 |
| 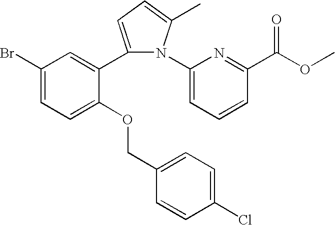 | 6-{2-[5-Bromo-2-(4- chloro-benzyloxy)- phenyl]-5-methyl-pyrrol-1- yl}-picolinic acid methyl ester | t = 4.07 [MH⁺]511/513/515 |
| 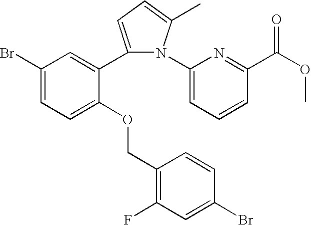 | 6-{2-[5-Bromo-2-(2-fluoro- 4-bromo-benzyloxy)- phenyl]-5-methyl-pyrrol-1- yl}-picolinic acid methyl ester | t = 4.15 [MH⁺]573/575/577 |
| 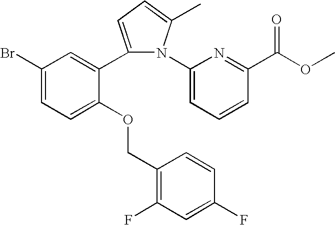 | 6-{2-[5-Bromo-2-(2,4- difluoro-benzyloxy)- phenyl]-5-methyl-pyrrol-1- yl}-picolinic acid methyl ester | t = 3.95 [MH⁺] 513/515 |
| 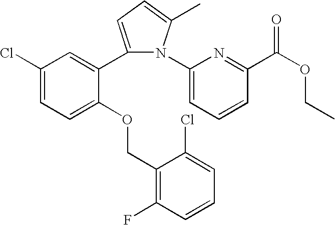 | 6-{2-[5-Chloro-2-(2- chloro-6-fluoro- benzyloxy)-phenyl]-3- methyl-pyrrol-1-yl}- picolinic acid ethyl ester | t = 3.99 min [MH⁺]499/501/503 |
| 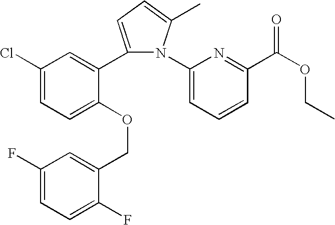 | 6-{2-[5-Chloro-2-(2,5- difluoro-benzyloxy)- phenyl]-5-methyl-pyrrol-1- yl}-picolinic acid ethyl ester | t = 3.98 [MH⁺] 483/485 |
| 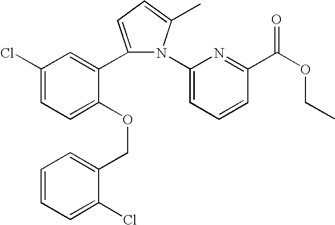 | 6-{2-[5-Chloro-2-(2- chloro-benzyloxy)- phenyl]-5-methyl-pyrrol-1- yl}-picolinic acid ethyl ester | t = 4.07 min [MH⁺]481/483/485 |
| 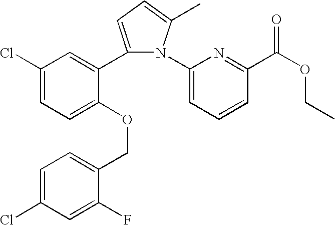 | 6-{2-[5-Chloro-2-(2-fluoro- 4-chloro-benzyloxy)- pheny]-5-methyl-pyrrol-1- yl}-picolinic acid ethyl ester | t = 4.09 min [MH⁺]499/501/503 |
| 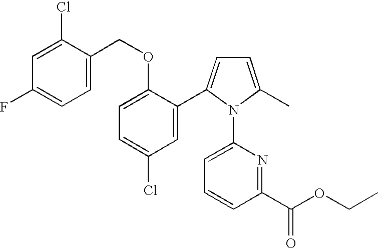 | 6-{2-[5-Chloro-2-(2- chloro-4-fluoro- benzyloxy)-phenyl]-5- methyl-pyrrol-1-yl}- picolinic acid ethyl ester | t = 4.19 min [MH⁺]499/501/503 |
| 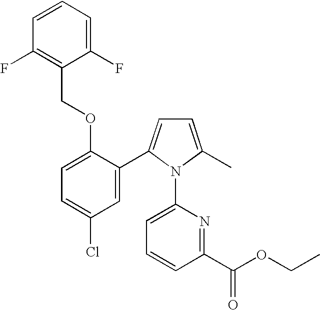 | 6-{2-[5-Chloro-2-(2,6- difluoro-benzyloxy)- phenyl]-5-methyl-pyrrol-1- yl}-picolinic acid ethyl ester | t = 3.91 min [MH⁺]483/485 |
| 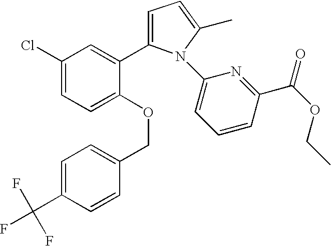 | 6-{2-[5-Chloro-2-(4- trifluoromethyl- benzyloxyl)-phenyl]-5- methyl-pyrrol-1-yl}- picolinic acid ethyl ester | t = 4.01 min [MH⁺]515/517 |
| 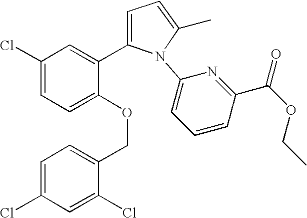 | 6-{2-[5-Chloro-2-(2,4- dichloro-benzyloxy)- phenyl]-5-methyl-pyrrol-1- yl}-picolinic acid ethyl ester | t = 4.27 [MH⁺]515/517/519/521 |
| 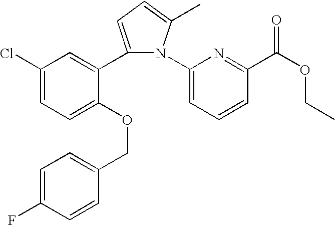 | 6-{2-[5-Chloro-2-(4-fluoro- benzyloxy)-phenyl]-5- ethyl-pyrrol-1-yl}- picolinic acid ethyl ester | t = 3.96 min [MH⁺]465/467 |
| 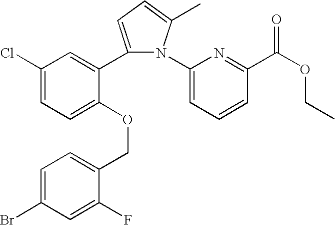 | 6-{2-[5-Chloro-2-(2-fluoro- 4-bromo-benzyloxy)- phenyl]-5-methyl-pyrrol-1- yl}-picolinic acid ethyl ester | t = 4.14 min [MH⁺]543/545/547 |
| 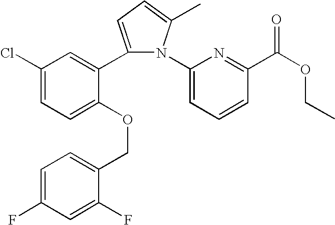 | 6-{2-[5-Chloro-2-(2,4- difluoro-benzyloxy)- phenyl]-5-methyl-pyrrol-1- yl}-picolinic acid ethyl ester | t = 3.99 [MH⁺] 483/485 |
| 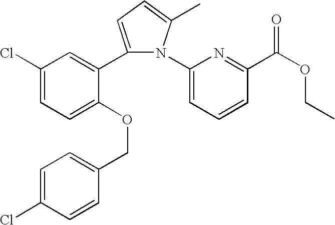 | 6-{2-[5-Chloro-2-(4- chloro-benzyloxy)- phenyl]-5-methyl-pyrrol-1- yl}-picolinic acid ethyl ester | t = 4.09 min [MH⁺]481/483/485 |
| 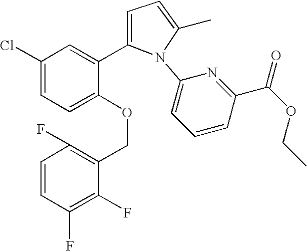 | 6-{2-[5-Chloro-2-(2,3,6- trifluoro-benzyloxy)- phenyl]-5-methyl-pyrrol-1- yl}-picolinic acid ethyl ester | t = 3.84 [MH⁺] 501/503 |
| 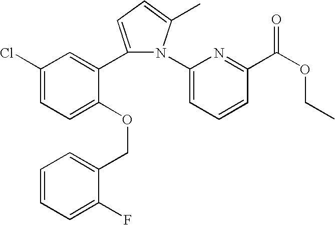 | 6-{2-[5-Chloro-2-(2-fluoro- benzyloxy)-phenyl]-5- methyl-pyrrol-1-yl}- picolinic acid ethyl ester | t = 3.96 [MH⁺] 465/467 |
| 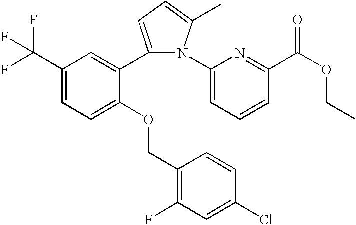 | 6-{2-[5-Trifluoromethyl-2- (2-fluoro-4-chloro- benzyl-phenyl]-5- methyl-pyrrol-1-yl}- picolinic acid ethyl ester | t = 4.14 [MH⁺] 533/535 |
| 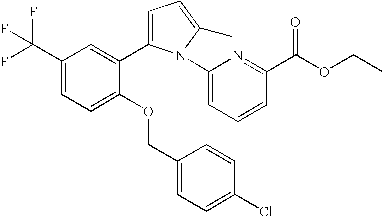 | 6-{2-[5-Trifluoromethyl-2- (2-chloro-benzyloxy)- phenyl]-5-methyl-pyrrol-1- yl}-picolinic acid ethyl ester | t = 4.13 [MH⁺] 515/517 |
| 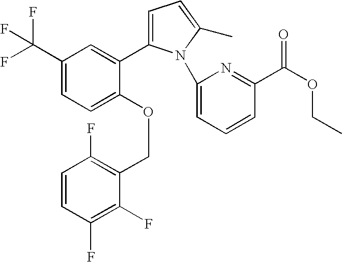 | 6-{2-[5-Trifluoromethyl-2- (2,3,6-trifluoro- benzyloxy)-phenyl]-5- methyl-pyrrol-1-yl}- picolinic acid ethyl ester | t = 3.96 [MH⁺] 535 |
| 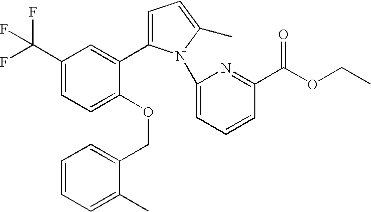 | 6-{2-[5-Trifluoromethyl-2- (2-methyl-benzyloxy)- phenyl]-5-methyl-pyrrol-1- yl}-picolinic acid ethyl ester | t = 4.08 [MH⁺] 495 |
| 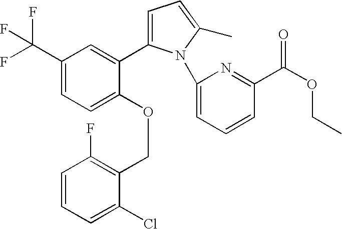 | 6-{2-[5-Trifluoromethyl-2- (2-chloro-6-fluoro- benzyloxy)-phenyl]-5- methyl-pyrrol-1-yl}- picolinic acid ethyl ester | t = 4.08 [MH⁺] 535/533 |
| 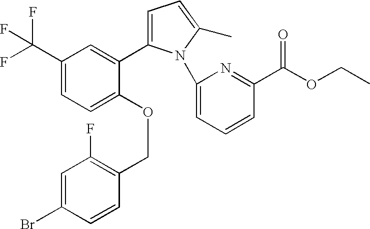 | 6-{2-[5-Trifluoromethyl-2- (2-fluoro-4-bromo- benzyloxy)-phenyl]-5- methyl-pyrrol-1-yl}- picolinic acid ethyl ester | t = 4.08 [MH⁺] 577/579 |
| 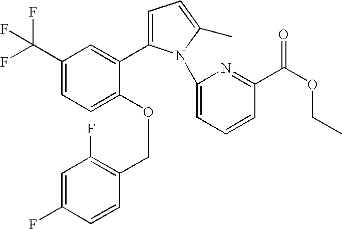 | 6-{2-[5-Trifluoromethyl-2- (2,4-difluoro-benzyloxy)- phenyl]-5-methyl-pyrrol-1- yl}-picolinic acid ethyl ester | t = 4.10 [MH⁺] 517 |
| 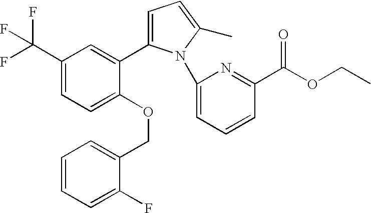 | 6-{2-[5-Trifluoromethyl-2- (2-fluoro-benzyloxy)- phenyl]-5-methyl-pyrrol-1- yl}-picolinic acid ethyl ester | t = 3.99 [MH⁺] 499 |
| 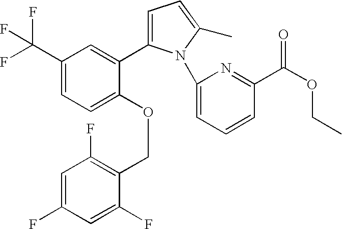 | 6-{2-[5-Trifluoromethyl-2- (2,4,6-trifluoro- benzyloxy)-phenyl]-5- methyl-pyrrol-1-yl}- picolinic acid ethyl ester | t = 3.99 [MH⁺] 535 |
| 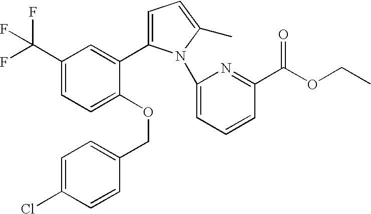 | 6-{2-[5-Trifluoromethyl-2- (4-chloro-benzyloxy)- phenyl]-5-methyl-pyrrol-1- yl}-picolinic acid ethyl ester | t = 4.14 [MH⁺] 515/517 |
| 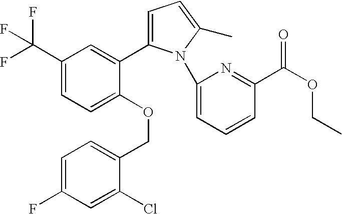 | 6-{2-[5-Trifluoromethyl-2- (2-chloro-4-fluoro- benzyloxy)-phenyl]-5- methyl-pyrrol-1-yl}- picolinic acid ethyl ester | t = 4.18 [MH⁺] 533/535 |
| 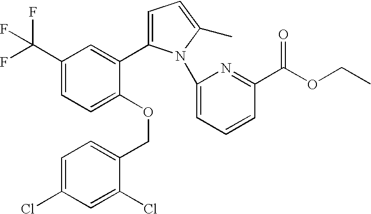 | 6-{2-[5-Trifluoromethyl-2- (2,4-dichloro-benzyloxy)- phenyl]-5-methyl-pyrrol-1- yl}-picolinic acid ethyl ester | t = 4.34 [MH⁺]549/551/553 |

### Example 21

### 6-{2-[5-Bromo-2-benzyloxy-phenyl]-5-methyl-pyrrol-1-yl}-picolinic acid

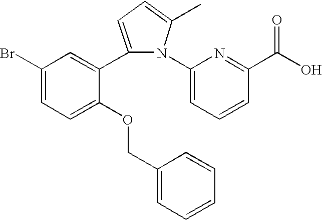

6-{2-[5-Bromo-2-benzyloxy-phenyl]-5-methyl-pyrrol-1-yl}-picolinic acid (150 mg) was dissolved in ethanol (5 ml) and 2M NaOH (1 ml) and was heated in a sealed vessel at 120° C. for 15 minutes using microwaves. Upon cooling the reaction was diluted with CH₂Cl₂ (20 ml) and shaken with dil. HCl (3 ml). The organics were separated using a phase separator column with a NaSO₄ cartridge attached and concentrated to give the title compound. LCMS t=3.97 min [MH⁺} 463/465

The following acids were prepared by a similar route to 6-{2-[5-bromo-2-(4-fluoro-benzyloxy)-phenyl]-5-methyl-pyrrol-1-yl}-picolinic-acid from the appropriate intermediates.

| | | | LCMS/NMR |
|---|---|---|---|
| Examples | Structure | Name | Data |
| 22 | 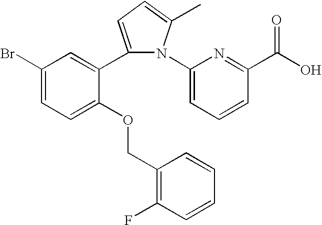 | 6-{2-[5-Bromo-2-(2- fluoro-benzyloxy)- phenyl]-5-methyl- pyrrol-1-yl}-picolinic acid | t = 3.98 min [MH⁺}481/483 |
| 23 | 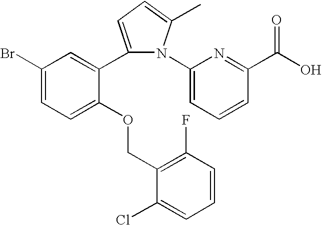 | 6-{2-[5-Bromo-2-(2- chloro-6-fluoro- benzyloxy)-phenyl]- 5-methyl-pyrrol-1- yl}-picolinic acid | t = 3.93 min [MH⁺}515/517/519 |
| 24 | 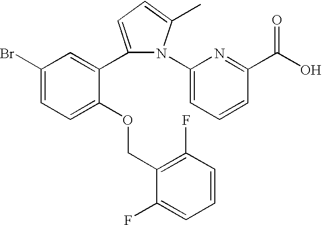 | 6-{2-[5-Bromo-2- (2,6-difluoro- benzyloxy)-phenyl]- 5-methyl-pyrrol-1- yl}-picolinic acid | t = 3.82 min [MH⁺}499/501 ¹H NMR (400 MHz, CDCl₃) 2.28(3H, s), 4.66(2H, s), 6.11 (1H, d, J=3Hz), 6.26 (1H, d, J=3Hz), 6.77 (1H, d, J=8Hz), 6.83-6.90(2H, m), 7.06(1H, d, J=8Hz), 7.27-7.37(2H, m), 7.47(1H, apparent s), 7.76(1H, t, J=8Hz), 8.01(1H, d, J=8Hz) |
| 25 | 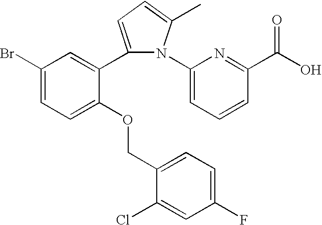 | 6-{2-[5-Bromo-2-(2- chloro-4-fluoro- benzyloxy)-phenyl]- 5-methyl-pyrrol-1- yl}-picolinic acid | t = 4.08 min [MH⁺}515/517/519 |
| 26 | 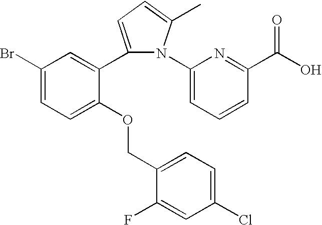 | 6-{2-[5-Bromo-2-(2- fluoro-4-chloro- benzyloxy)-phenyl]- 5-methyl-pyrrol-1- yt}-picolinic acid | t = 4.06 min [MH⁺}515/517/519 |
| 27 | 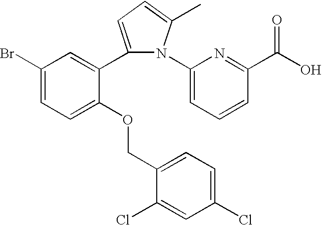 | 6-{2-[5-Bromo-2- (2,4-dichloro- benzyloxy)-phenyl]- 5-methyl-pyrrol-1- yl}-picolinic acid | t = 4.26 min [MH⁺}531/533/535/537 |
| 28 | 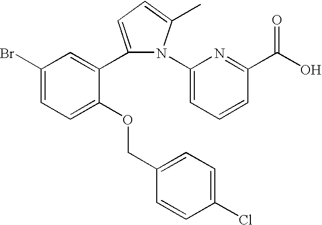 | 6-{2-[5-Bromo-2-(4- chloro-benzyloxy)- phenyl]-5-methyl- pyrrol-1-yl}-picolinic acid | t = 4.04 min [MH⁺}497/499/501 |
| 29 | 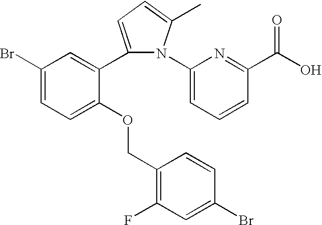 | 6-{2-[5-Bromo-2-(2- fluoro-4-bromo- benzyloxy)-phenyl]- 5-methyl-pyrrol-1- yl}-picolinic acid | t = 4.24 min [MH⁺}559/561/563 ¹H NMR (400 MHz, CDCl₃) 2.33(3H, s), 4.58(2H, s), 6.17(1H, d, J=3Hz), 6.31(1H, d, J=3Hz), 6.61(1H, d, J=8Hz), 6.88(1H, t, J=8Hz), 7.09(1H, d, J=8Hz), 7.18-7.24(2H, m), 7.28-7.34(1H, m), 7.48(1H, apparent s), 7.78(1H, t, |
| # J=8Hz), 8.03(1H, d, J=8Hz) | | | |
| 30 | 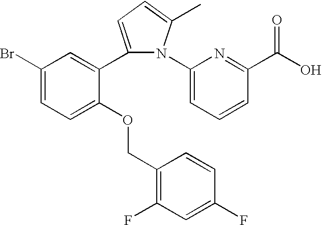 | 6-{2-[5-Bromo-2- (2,4-difluoro- benzyloxy)-phenyl]- 5-methyl-pyrrol-1- yl}-picolinic acid | t = 3.99 min [MH⁺}499/501 |
| 31 | 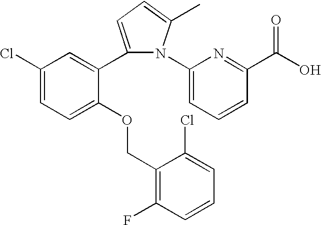 | 6-{2-[5-Chloro-2-(2- chloro-6-fluoro- benzyloxy)-phenyl]- 5-methyl-pyrrol-1- yl}-picolinic acid | t = 3.88 min [MH⁺]471/473/475 |
| 32 | 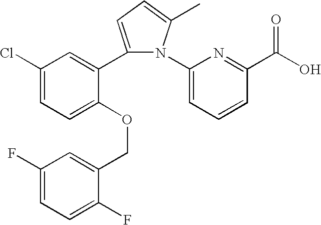 | 6-{2-[5-Chloro-2- (2,5-difluoro- benzyloxy)-phenyl]- 5-methyl-pyrrol-1- yl}-picolinic acid | t = 3.88 min [MH⁺]455/457 |
| 33 | 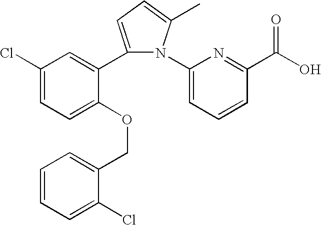 | 6-{2-[5-Chloro-2-(2- chloro-benzyloxy)- phenyl]-5-methyl- pyrrol-1-yl}-picolinic acid | t = 4.03 min [MH⁺]453/455/457 |
| 34 | 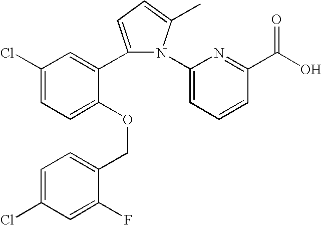 | 6-{2-[5-Chloro-2-(2- fluoro-4-chloro- benzyloxy)-phenyl]- 5-methyl-pyrrol-1- yl}-picolinic acid | t = 4.09 min [MH⁺]471/473/475 |
| 35 | 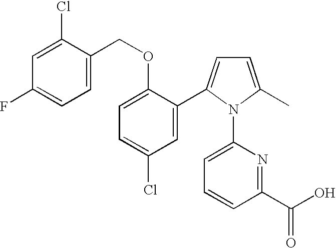 | 6-{2-[5-Chloro-2-(2- chloro-4-fluoro- benzyloxy)-phenyl]- 5-methyl-pyrrol-1- yl}-picolinic acid | t = 4.01 min [MH⁺]471/473/475 |
| 36 | 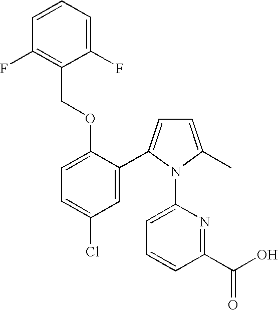 | 6-{2-[5-Chloro-2- (2,6-difluoro- benzyloxy)-phenyl]- 5-methyl-pyrrol-1- yl}-picolinic acid | t = 3.76 min [MH⁺]455/457 |
| 37 | 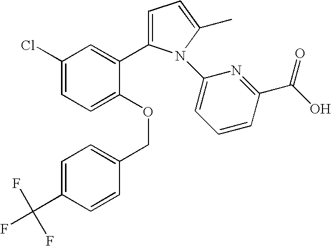 | 6-{2-[5-Chloro-2-(4- trifluoromethyl- benzyloxy)-phenyl]- 5-methyl-pyrrol-1- yl}-picolinic acid | t = 3.94 min [MH⁺]487/489 |
| 38 | 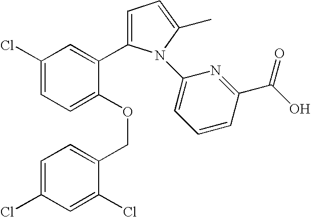 | 6-{2-[5-Chloro-2- (2,4-dichloro- benzyloxy)-phenyl]- 5-methyl-pyrrol-1- yl}-picolinic acid | t = 4.19 min [MH⁺]487/489/491/493 |
| 39 | 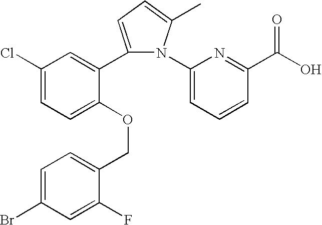 | 6-{2-[5-Chloro-2-(2- fluoro-4-bromo- benzyloxy)-phenyl]- 5-methyl-pyrrol-1- yl}-picolinic acid | t = 4.05 min [MH⁺]515/517/519 |
| 40 | 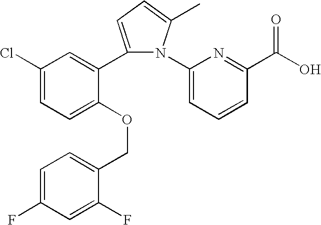 | 6-{2-[5-Chloro-2- (2,4-difluoro- benzyloxy)-phenyl]- 5-methyl-pyrrol-1- yl}-picolinic acid | t = 3.86 min [MH⁺]455/457 |
| 41 | 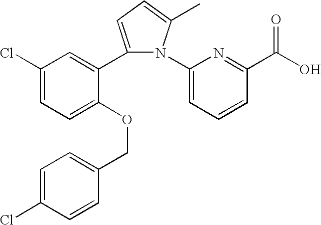 | 6-{2-[5-Chloro-2-(4- chloro-benzyloxy)- phenyl]-5-methyl- pyrrol-1-yl}-picolinic acid | t = 3.98 min [MH⁺]453/455/457 |
| 42 | 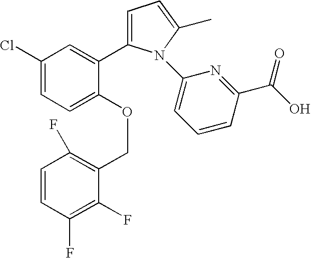 | 6-{2-[5-Chloro-2- (2,3,6-trifluoro- benzyloxy)-phenyl]- 5-methyl-pyrrol-1- yl}-picolinic acid | t = 3.75 min [MH⁺]473/475 |
| 43 | 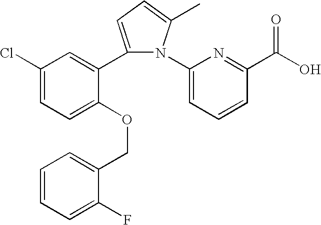 | 6-{2-[5-Chloro-2-(2- fluoro-benzyloxy)- phenyl]-5-methyl- pyrrol-1-yl}-picolinic acid | t = 3.85 min [MH⁺]437/439 |
| 44 | 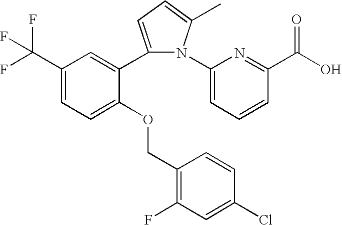 | 6-{2-[5- Trifluoromethyl-2- (2-fluoro-4-chloro- benzyloxy)-phenyl]- 5-methyl-pyrrol-1- yl}-picolinic acid | t = 4.00 min [MH⁺]505/507 |
| 45 | 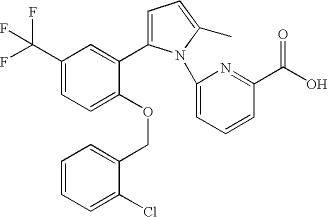 | 6-{2-[5- Trifluoromethyl-2- (2-chloro- benzyloxy)-phenyl]- 5-methyl-pyrrol-1- yl}-picolinic acid | t = 4.09 min [MH⁺]487/489 |
| 46 | 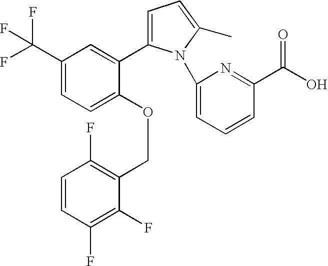 | 6-{2-[5- Trifluoromethyl-2- (2,3,6-trifluoro- benzyloxy)-phenyl]- 5-methyl-pyrrol-1- yl}-picolinic acid | t = 3.81 min [MH⁺]507 |
| 47 | 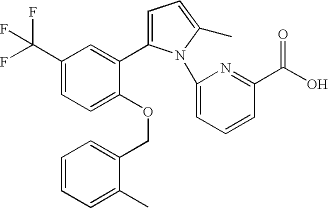 | 6-{2-[5- Trifluoromethyl-2- (2-methyl- benzyloxy)-phenyl]- 5-methyl-pyrrol-1- yl}-picolinic acid | t = 3.98 min [MH⁺]467 |
| 48 | 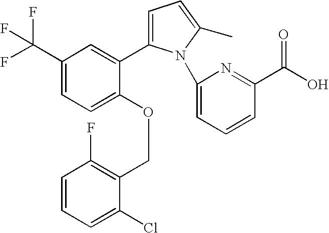 | 6-{2-[5- Trifluoromethyl-2- (2-chloro-6-fluoro- benzyloxy)-phenyl]- 5-methyl-pyrrol-1- yl}-picolinic acid | t = 3.92 min [MH⁺]505/507 |
| 49 | 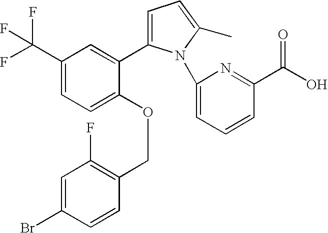 | 6-{2-[5- Trifluoromethyl-2- (2-fluoro-4-bromo- benzyloxy)-phenyl]- 5-methyl-pyrrol-1- yl}-picolinic acid | t = 4.13 min [MH⁺]549/551 |
| 50 | 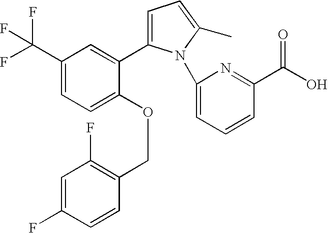 | 6-{2-[5- Trifluoromethyl-2- (2,4-difluoro- benzyloxy)-phenyl]- 5-methyl-pyrrol-1- yl}-picolinic acid | t = 3.90 min [MH⁺]489 |
| 51 | 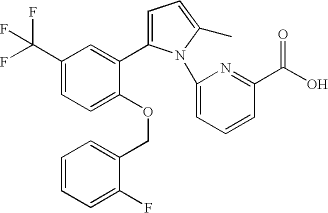 | 6-{2-[5- Trifluoromethyl-2- (2-fluoro- enzyloxy)-phenyl]- 5-methyl-pyrrol-1- yl}-picolinic acid | t = 3.88 min [MH⁺]471 ¹H NMR (400 MHz, CDCl₃) 2.32(3H, s), 4.74(2H, s), 6.09(1H, d, J=3Hz), 6.38(1H, d, J=3Hz), 6.84(1H, d, J=8Hz), 7.00-7.12(4H, m), 7.27(1H, m), 7.48 (1H, dd, J=8Hz, 2 Hz), 7.60(1H, d, J=2Hz), 7.77(1H, t, J=8Hz), 8.02(1H, d, J=8Hz) |
| 52 | 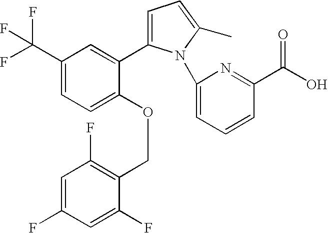 | 6-{2-[5- Trifluoromethyl-2- (2,4,6-trifluoro- benzyloxy)-phenyl]- 5-methyl-pyrrol-1- yl}-picolinic acid | t = 3.85 min [MH⁺]507 ¹H NMR (400 MHz, CDCl₃) 2.29(3H, s), 4.72(2H, s), 6.14(1H, d, J=3Hz), 6.32(1H, d, J=3Hz), 6.63-6.71(2H, m), 6.95(1H, d, J=8Hz), 7.48-7.55(2H, m), 7.27(1H, m), 7.81 (1H, t, J=8Hz), 8.04 (1H, d, J=8Hz) |
| 53 | 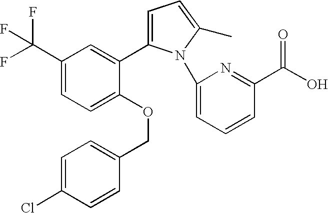 | 6-{2-[5- Trifluoromethyl-2- (4-chloro- benzyloxy)-phenyl]- 5-methyl-pyrrol-1- yl}-picolinic acid | t = 4.06 min [MH⁺]487/489 |
| 54 | 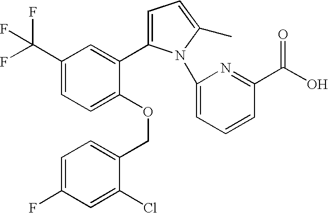 | 6-{2-[5- Trifluoromethyl-2- (2-chloro-4-fluoro- benzyloxy)-phenyl]- 5-methyl-pyrrol-1- yl}-picolinic acid | t = 4.04 min [MH⁺]505/507 |
| 55 | 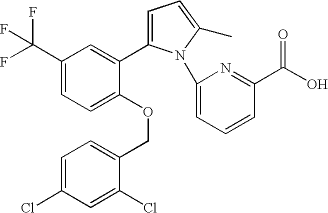 | 6-{2-[5- Trifluoromethyl-2- (2,4-dichloro- benzyloxy)-phenyl]- 5-methyl-pyrrol-1- yl}-picolinic acid | t = 4.26 min [MH⁺]521/523/524 |
| 56 | 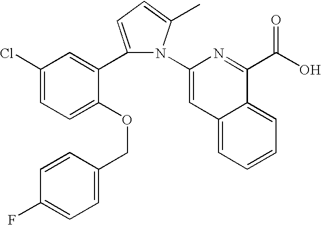 | 3-{2-[5-Chloro-2-(4- fluoro-benzyloxy)- phenyl]-5-methyl- pyrrol-1-yl]- isoquinoline-1- carboxylic acid | t = 4.40 [MH⁺]487/489 |

### 3-[2-(5-Chloro-2-isobutyloxy-phenyl)-5-methyl-pyrrol-1-yl]-6-methyl benzoic acid ethyl ester

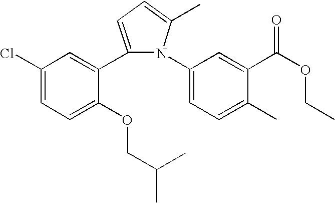

A solution of 1-[5-chloro-2-isobutyloxyphenyl]-pentane-1,4-dione (0.065 g, 0.23 mmol), ethyl-3-amino-6-methyl-benzoate (0.041 g, 0.23 mmol) and pTSA (cat) in anhydrous acetonitrile (1 mL) were heated in a microwave reactor for 10 minutes at 160° C. The reaction mixture was diluted with CH₂Cl₂ (10 mL) and shaken with 2N HCl (1 mL), organics separated, dried (MgSO₄) and concentrated in vacuo. The oil was purified by silica chromatography (cyclohexane to 20% EtOAc in cyclohexane) to yield the title compound as a yellow oil (0.0589, 60%). LCMS rt=4.56 min [MH⁺] 426/428

The following esters were prepared by a similar route to 3-[2-(5-chloro-2-isobutyloxy-phenyl)-5-methyl-pyrrol-1-yl]-6-methyl benzoic acid ethyl ester from the appropriate intermediates.

| Structure | Name | LCMS |
|---|---|---|
| 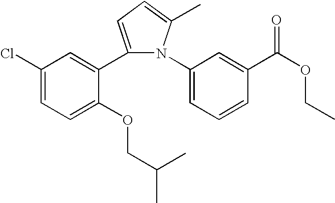 | 3-[2-(5-Chloro-2- isobutyloxy-phenyl)-5- methyl-pyrrol-1-yl]- benzoic acid ethyl ester | t = 4.47 [MH⁺] 412/414 |
| 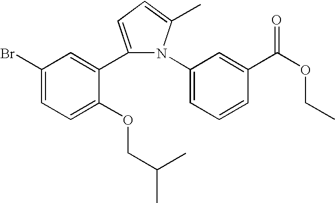 | 3-[2-(5-Bromo-2- isobutyloxy-phenyl)-5- methyl-pyrrol-1-yl]- benzoic acid ethyl ester | t = 4.51 [MH⁺] 456/458 |
| 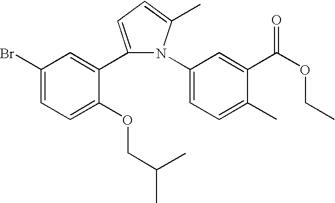 | 3-[2-(5-Bromo-2- isobutyloxy-phenyl)-5- methyl-pyrrol-1-yl]-6- methyl-benzoic acid ethyl ester | t = 4.61 [MH⁺] 470/472 |
| 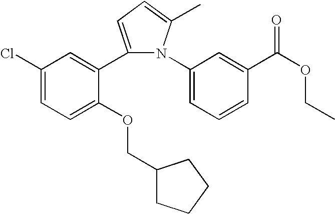 | 3-[2-(5-Chloro-2- cyclopentylmethoxy- phenyl)-5-methyl-pyrrol-1- yl]-benzoic acid ethyl ester | |
| 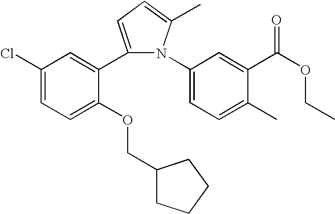 | 3-[2-(5-Chloro-2- cyclopentylmethoxy- phenyl)-5-methyl-pyrrol-1- yl]-6-methyl-benzoic acid ethyl ester | |
| 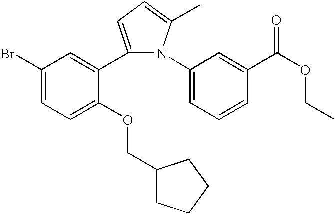 | 3-[2-(5-Bromo-2- cyclopentylmethoxy- phenyl)-5-methyl-pyrrol-1- yl]-benzoic acid ethyl ester | |
| 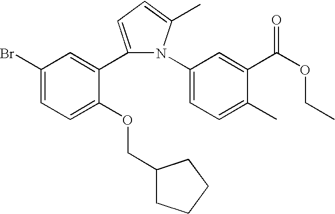 | 3-[2-(5-Bromo-2- cyclopentylmethoxy- phenyl)-5-methyl-pyrrol-1- yl]-6-methyl-benzoic acid ethyl ester | |

### Example 57

### 3-[2-(5-Chloro-2-isobutyloxy-phenyl)-5-methyl-pyrrol-1-yl]-6-methyl benzoic acid sodium salt

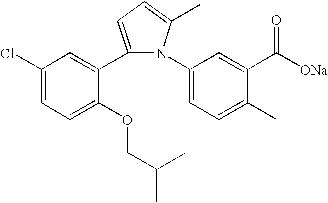

3-[2-(5-Chloro-2-isobutyloxy-phenyl)-5-methyl-pyrrol-1-yl]-6-methyl benzoic acid ethyl ester (58 mg) was dissolved in ethanol (2 ml) and 2M NaOH (1 ml) and was heated in a sealed vessel at 100° C. for 5 minutes using microwaves. Upon cooling the reaction was diluted with CH₂Cl₂ (20 ml) and shaken with dil. HCl (3 ml). The organics were separated using a phase separator column with a NaSO₄ cartridge attached and concentrated. The oil was purified by silica chromatography (cyclohexane to 20% EtOAc in cyclohexane) to yield the acid. This was dissolved in methanol (5 ml) and 2M NaOH (1 ml) and was heated in a sealed vessel at 100° C. for 5 minutes using microwaves. Upon cooling, the mixture was concentrated in vacuo, the resulting residue diluted with water and extracted with EtOAc (x3), the organics dried (Na₂SO₄), filtered and concentrated to give the title compound (48 mg, 84%). LCMS t=4.23 min [MH⁺] 398/400

The following salts were prepared by a similar route to 3-[2-(5-Chloro-2-isobutyloxy-phenyl)-5-methyl-pyrrol-1-yl]-6-methyl benzoic acid sodium salt from the appropriate intermediates.

| Examples | Structure | Name | LCMS |
|---|---|---|---|
| 58 | 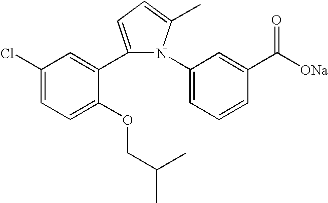 | 3-[2-(5-Chloro-2- isobutyloxy-phenyl)- 5-methyl-pyrrol-1- yl]-benzoic acid sodium salt | t = 4.12 [MH⁺]384/386 |
| 59 | 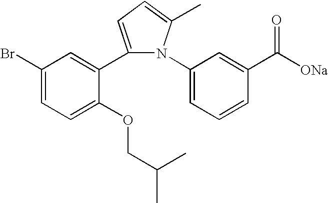 | 3-[2-(5-Bromo-2- isobutyloxy-phenyl)- 5-methyl-pyrrol-1- yl]-benzoic acid sodium salt | t = 4.19 [MH⁺]428/430 |
| 60 | 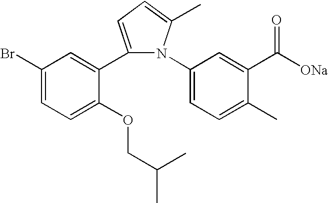 | 3-[2-(5-Bromo-2- isobutyloxy-phenyl)- 5-methyl-pyrrol-1- yl]-6-methyl-benzoic acid sodium salt | t = 4.25 [MH⁺]442/444 |
| 61 | 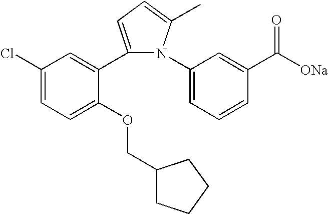 | 3-[2-(5-Chloro-2- cyclopentylmethoxy- phenyl)-5-methyl- pyrrol-1-yl]-benzoic acid sodium salt | t = 4.36 [MH⁺]410/412 |
| 62 | 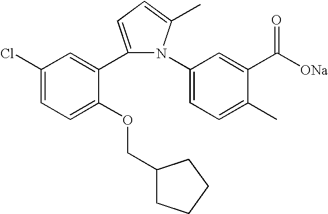 | 3-[2-(5-Chloro-2- cyclopentylmethoxy- phenyl)-5-methyl- pyrrol-1-yl]-6- methyl-benzoic acid sodium salt | t = 4.41 [MH⁺]424/426 |
| 63 | 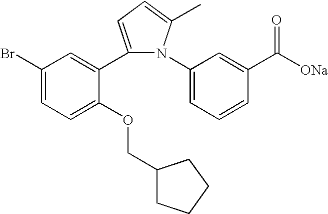 | 3-[2-(5-Bromo-2- cyclopentylmethoxy- phenyl)-5-methyl- pyrrol-1-yl]-benzoic acid sodium salt | t = 4.41 [MH⁺]454/456 |
| 64 | 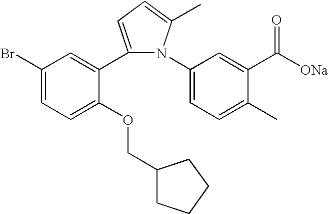 | 3-[2-(5-Bromo-2- cyclopentylmethoxy- phenyl)-5-methyl- pyrrol-1-yl]-6- methyl-benzoic acid sodium salt | t = 4.46 [MH⁺]468/470 |

### Example 65

### 3-[2-(5-Chloro-2-isobutyloxy-phenyl)-5-methyl-pyrrol-1-yl]-6-fluoro benzoic acid sodium salt

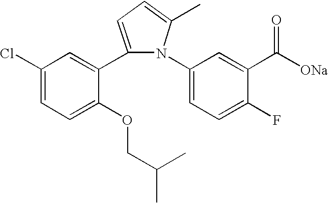

A solution of 1-[5-chloro-2-isobutyloxyphenyl]-pentane-1,4-dione (0.25 g, 0.88 mmol), 3-amino-6-fluoro-benzoic acid (0.14 g, 0.88 mmol) and pTSA (cat) in anhydrous acetonitrile (5 mL) were heated in a microwave reactor for 10 minutes at 160° C. Upon cooling the reaction was diluted with CH₂Cl₂ (20 ml) and shaken with dil. HCl (3 ml). The organics were separated using a phase separator column with a NaSO₄ cartridge attached and concentrated. The oil was purified by silica chromatography (cyclohexane to 20% EtOAc in cyclohexane) to yield the acid. This was dissolved in methanol (5 ml) and 2M NaOH (1 ml) and was heated in a sealed vessel at 100° C. for 5 minutes using microwaves. Upon cooling, the mixture was concentrated in vacuo, the resulting residue diluted with water and extracted with EtOAc (x3), the organics dried (Na₂SO₄), filtered and concentrated to give the title compound (0.14 g, 38%). LCMS rt=4.13 min [MH⁺] 402/404

The following salts were prepared by a similar route to 3-[2-(5-chloro-2-isobutyloxy-phenyl)-5-methyl-pyrrol-1-yl]-6-fluoro benzoic acid sodium salt from the appropriate intermediates.

| Examples | Structure | Name | LCMS |
|---|---|---|---|
| 66 | 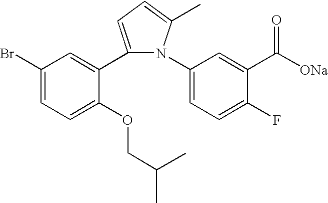 | 3-[2-(5-Bromo-2- isobutyloxy-phenyl)- 5-methyl-pyrrol-1- yl]-6-fluoro-benzoic acid sodium salt | t = 4.19 [MH⁺]446/448 |
| 67 | 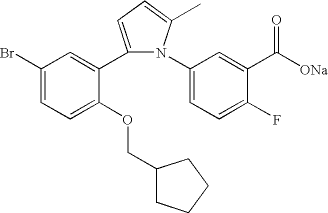 | 3-[2-(5-Bromo-2- cyclopentylmethoxy- phenyl)-5-methyl- pyrrol-1-yl]-6-fluoro- benzoic acid sodium salt | t = 4.47 [MH⁺]472/474 |
| 68 | 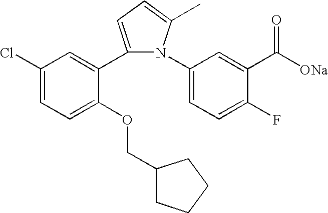 | 3-[2-(5-Chloro-2- cyclopentylmethoxy- phenyl)-5-methyl- pyrrol-1-yl]-6-fluoro- benzoic acid sodium salt | t = 4.40 [MH⁺]428/430 |

### 5-Chloro-2-methoxy-benzoyl chloride

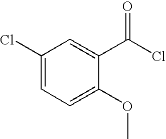

Thionyl chloride (7.8 mL, 0.108mol) was added dropwise to a solution of 5-chloro-2-methoxy-benzoic acid (10 g, 0.054mol) in anhydrous CH₂Cl₂ (54 mL). The reaction was heated to reflux and stirred under nitrogen for 23 hours. The reaction mixture was allowed to cool and reduced in vacuo. The acid chloride was used immediately in the next stage.

The following acid chlorides were prepared by a similar route to 5-chloro-2-methoxy-benzoyl chloride from the appropriate intermediates and used immediately in their respective next stage.

### 1-(5-Chloro-2-methoxy-phenyl)-3-[1,3]dioxolan-2-yl-propan-1-one

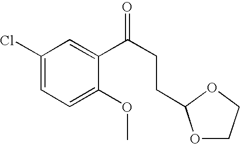

| Structure | Name |
|---|---|
| 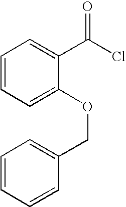 | 2-Benzyloxy-benzoyl chloride |
| 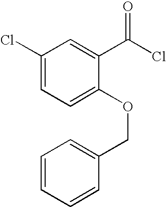 | 2-Benzyloxy-5-chloro-benzoyl chloride |
| 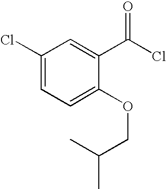 | 5-Chloro-2-isobutyloxy-benzoyl chloride |
| 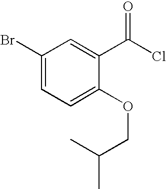 | 5-Bromo-2-isobutyloxy-benzoyl chloride |

2-(2-bromoethyl)-1,3-dioxolane (0.008 mol, 1 mL) was added dropwise to a suspension of magnesium turnings (0.113 mol, 2.73 g) in anhydrous THF (40 mL) under nitrogen. The reaction was heated to 50° C. before the dropwise addition of further 2-(2-bromoethyl)-1,3-dioxolane (0.048 mol, 5.61 mL), maintaining a constant reflux. After complete addition the reaction was cooled to 0° C., and then transferred via a cannula over a period of 25 minutes to a pre-cooled (−65° C.) solution of 5-chloro-2-methoxy-benzoyl chloride (0.054 mol) in anhydrous THF (40 mL), maintaining the reaction temperature in the range −45° C. to −65.

After complete addition, the reaction was allowed to slowly rise in temperature to −4° C. over a period of 2 hours, before the addition of water (20 mL). The aqueous layer was separated, and extracted with EtOAc (2×100 mL). The extracts were combined with the organic layer, dried (MgSO₄), filtered and concentrated in vacuo. The oil was purified by flash silica column chromatography (cyclohexane to 15% EtOAc in cyclohexane) to afford the title compound as a cream solid (1.58 g, 11%).

¹H NMR (CDCl₃) −δ 2.08 (dt, 2H), 3.09 (t, 2H), 3.90 (m, 7H), 6.90 (d, 1H), 7.39 (dd, 1H), 7.65 (d 1H).

The following dioxolanes were prepared by a similar route to 1-(5-chloro-2-methoxy-phenyl)-3-[1,3]dioxolan-2-yl-propan-1-one from the appropriate intermediates.

| Structure | Name | Data |
|---|---|---|
| 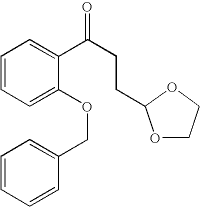 | 1-(2-Benzyloxy-phenyl)-3- [1,3]dioxolan-2-yl-propan- 1-one | ¹H NMR (CDCl₃) δH 2.00-2.10(2H, m), 3.12 (2H , t), 3.74-3.87(4H, m), 4.99(1H, t), 5.16(2H, s), 6.95(2H, m), 7.29-7.56(6H, m), 7.70 (1H, dd). |
| 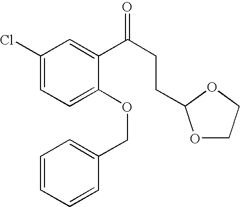 | 1-(2-Benzyloxy-5-chloro- phenyl)-3-[1,3]dioxolan-2- yl-propan-1-one | |
| 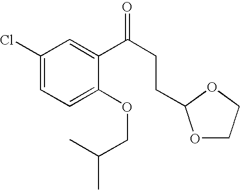 | 1-(5-Chloro-2-isobutyloxy- phenyl)-3-[1,3]dioxolan-2- yl-propan-1-one | LCMS rt = 4.39 [MH⁺]313/315 |
| 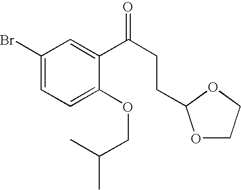 | 1-(5-Bromoo-2- isobutyloxy-phenyl)-3- [1,3]dioxolan-2-yl-propan- 1-one | LCMS rt = 4.46 [MH⁺]357/359 |

### 3-[2-(5-Chloro-2-methoxy-phenyl)-pyrrol-1-yl]-benzoic acid ethyl ester

A solution of 1-(5-chloro-2-methoxy-phenyl)-3-[1,3]dioxolan-2-yl-propan-1-one (1.58 g, 5.82 mmol), ethyl-3-aminobenzoate (6.4 mmol, 0.96 mL) and pTSA (cat) in anhydrous 1-methyl-2-pyrrolidinone (6 mL) were heated in a microwave reactor for 12 minutes at 150° C. The reaction mixture was diluted with EtOAc (100 mL) and washed with 2N HCl (25 mL) and saturated NaHCO₃ (25 mL), dried (MgSO₄) and concentrated in vacuo. The oil was purified by silica chromatography (cyclohexane to 5% EtOAc in cyclohexane) to yield the title compound as an orange oil (0.91 g, 44%).

¹H NMR (CDCl₃) −δ1.37 (t, 3H), 3.24 (s, 3H), 4.35 (q, 2H), 6.40 (m, 2H), 6.60 (d, 1H), 7.04 (dd, 1H), 7.20 (dd, 1H), 7.23 (ddd, 1H), 7.31 (dd, 1H), 7.35 (d, 1H), 7.89 (m, 2H).

The following pyrroles were prepared by a similar route to 3-[2-(5-chloro-2-methoxy-phenyl)-pyrrol-1-yl]-benzoic acid ethyl ester from the appropriate intermediates.

| Structure | Name | LCMS |
|---|---|---|
| 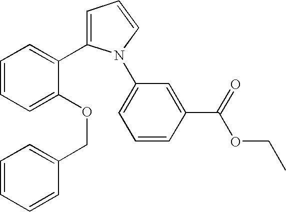 | 3-[2-(2-Benzyloxy- phenyl)-pyrrol-1-yl]- benzoic acid ethyl ester | t = 4.03 min [MH⁺] 398 |
| 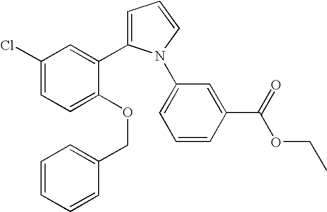 | 3-[2-(5-Chloro-2- benzyloxy-phenyl)-pyrrol- 1-yl]-benzoic acid ethyl ester | t = 4.03 min [MH⁺]432/434 |
| 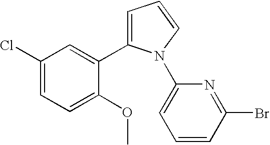 | 2-Bromo-6-[2-(5-chloro-2- methoxy-phenyl)-pyrrol-1- yl]-pyridine | t = 3.74 min [MH⁺]363/365/367 |
| 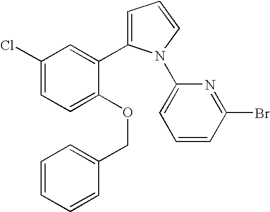 | 2-Bromo-6-[2-(5-chloro-2- benzyloxy-phenyl)-pyrrol- 1-yl]-pyridine | t = 3.97 min [MH⁺]440/441/443 |

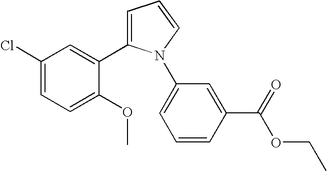

### 3-[2-(5-Chloro-2-methoxy-phenyl)-5-trifluoromethyl-pyrrol-1-yl]-benzoic add ethyl ester

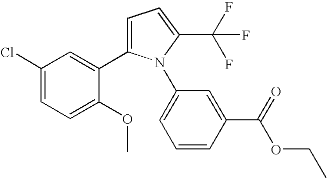

Iron (II) sulfate heptahydrate (1.12 mmol, 0.310 g) was added to a solution of 3-[2-(5-Chloro-2-methoxy-phenyl)-pyrrol-1-yl]-benzoic acid ethyl ester (0.66 g, 1.86 mmol) in anhydrous DMSO (10 mL). Trifluoromethyl iodide was bubbled through the reaction for two minutes. Hydrogen peroxide (30% wt/wt aqueous solution, 1.26 mL) was then added and the reaction was stirred at 22° C. for 2 hours. The reaction was added to saturated aqueous sodium sulfite solution (100 mL) and the suspension extracted with with diethyl ether (100 mL). The ether extraction was dried (MgSO₄) and and concentrated in vacuo. The crude product was purified by silica chromatography (cyclohexane to 5% EtOAc in cyclohexane) to yield the title compound as an yellow oil (0.34 g, 43%).

LCMS t=3.94 min [MH⁺] 424

### 3-[2-(5-Chloro-2-hydroxy-phenyl)-5-trifluoromethyl-pyrrol-1-yl]-benzoic acid

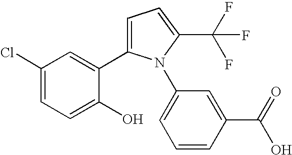

Sodium thiomethoxide (4 mmol, 0.28 g) was added to a solution of 3-[2-(5-Chloro-2-methoxy-phenyl)-5-trifluoromethyl-pyrrol-1-yl]-benzoic acid ethyl ester (0.80 mmol, 0.341 g) in anhydrous dimethylformamide (7.5 mL). The reaction was heated to 100° C. and stirred for 4 hours. The reaction mixture was partitioned between 2N HCl (30 mL) and EtOAc (50 mL). The aqueous layer was separated and extracted with EtOAc (2×50 mL). The EtOAc layers were combined, washed with brine (30 mL), dried (MgSO₄) and concentrated in vacuo. The crude product was purified by silica chromatography (Cyclohexane to CH₂Cl₂ to 30% EtOAc in CH₂Cl₂) to yield a yellow oil (0.26 g, 85%).

¹H NMR (CDCl₃) −δ 6.42 (d, 1H), 6.77 (d, 1H), 6.86 (d, 1H), 6.89 (d, 1H), 7.09 (dd, 1H), 7.44 (m, 2H), 7.98 (s, 1H), 8.02 (m, 2H), 8.09 (m, 1H).

### Example 69

### 3-{2-[5-Chloro-2-(2,4-difluoro-benzyloxy)-phenyl]-5-trifluoromethyl-pyrrol-1-yl}-benzoic acid

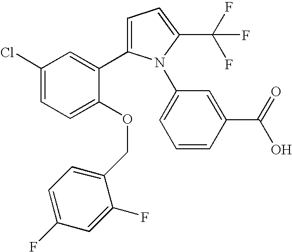

Potassium carbonate (0.37 mmol, 65 mg) and potassium iodide (0.034 mmol, 5.6 mg) was added to a solution of 3-[2-(5-chloro-2-hydroxy-phenyl)-5-trifluoromethyl-pyrrol-1-yl]-benzoic acid (0.17 mmol, 0.065 mg) in anhydrous MeOH (1.7 mL). The reaction was heated to 60° C. before the dropwise addition of 2,4-difluorobenzyl bromide (0.34 mmol, 44 μL). Heating was continued for one hour. The reaction was cooled, concetnrated in vacuo and partitioned between 2N HCl (2 mL) and CH₂Cl₂ (2 mL). The organic layer was collected and the solvent removed. The crude product was purified via MDAP to yield a white solid (42.3 mg, 49%). LCMS t=3.97 min [MS−] 506/508.

The following acids were prepared by a similar route to 3-{2-[5-chloro-2-(2,4-difluoro-benzyloxy)-phenyl]-5-trifluoromethyl-pyrrol-1-yl}-benzoic acidfrom the appropriate intermediates.

| Example | Structure | Name | LCMS |
|---|---|---|---|
| 70 | 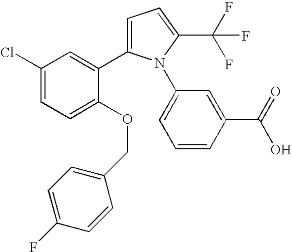 | 3-{2-[5-Chloro-2-(4- fluoro-benzyloxy)- phenyl]-5- trifluoromethyl-pyrrol-1- yl}-benzoic acid | t = 3.96 min [MS−]488/490 |
| 71 | 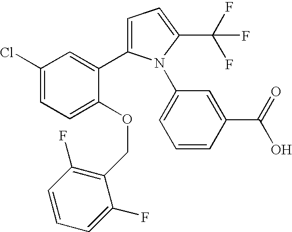 | 3-{2-[5-Chloro-2-(2,6- difluoro-benzyloxy)- phenyl]-5- trifluoromethyl-pyrrol-1- yl)-benzoic acid | t = 3.89 min [MS−]506/508 |
| 72 | 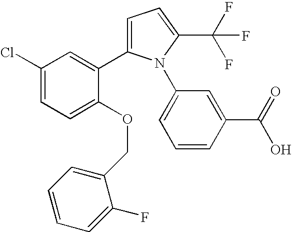 | 3-{2-[5-Chloro-2-(2- fluoro-benzyloxy)- phenyl]-5- trifluoromethyl-pyrrol-1- yl}-benzoic acid | t = 3.96 min [MS−]488/490 |
| 73 | 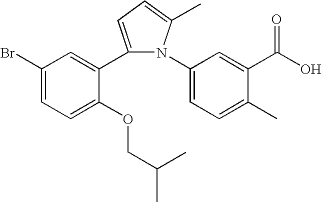 | 3-[2-(5-Bromo-2- isobutyloxy-phenyl)-5- methyl-pyrrol-1-yl-6- methyl benzoic acid | t = 4.29 min [MH+]442/444 |

### Example 74

### 3-[2-(2-Benzyloxy-phenyl)-pyrrol-1-yl]-benzoic acid

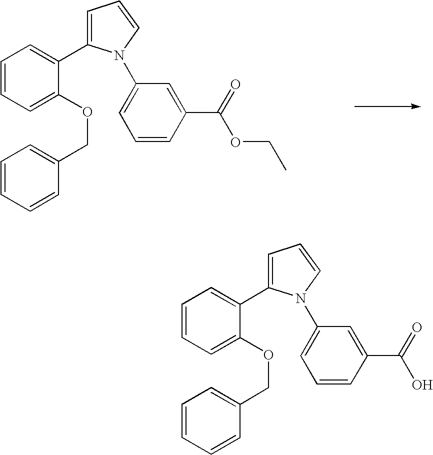

3-[2-(2-Benzyloxy-phenyl)-pyrrol-1-yl]-benzoic acid ethyl ester (0.046 g, 0.1 mmol) was heated in a mixture of ethanol (0.5 mL) and 2M sodium hydroxide (1.5 mL) at 100° C. in a microwave for 2 minutes. Upon cooling, the mixture was diluted with CH₂Cl₂ and 2M HCl then filtered through a hydrophobic frit, fitted with a sodium sulfate cartridge, and evaporated to give the title compound. LCMS t=3.78 min [MH⁺] 370; [MH−] 368.

The following acids were prepared by a similar route to 3-[2-(2-benzyloxy-phenyl)-pyrrol-1-yl]-benzoic acid from the appropriate intermediates.

| Example | Structure | Name | LCMS |
|---|---|---|---|
| 75 | 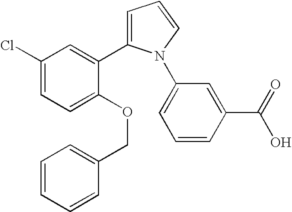 | 3-[2-(5-Chloro-2-benzyloxy- phenyl)-pyrrol-1-yl]-benzoic acid | t = 3.86 min [MH+] 404/406; [MH−] 402/404 |

### Example 76

### 3-[2-(5-Chloro-2-isobutyoxy-phenyl)-pyrrol-1-yl]-6-fluoro-benzoic acid sodium salt

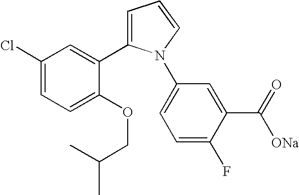

A solution of 1-(5-chloro-2-isobutyloxy-phenyl)-3-[1,3]dioxolan-2-yl-propan-1-one (0.1 g, 0.3 mmol), 3-amino-6-fluoro-benzoic acid (0.05 g, 0.3 mmol) and pTSA (cat) in anhydrous acetonitrile (1 mL) were heated in a microwave reactor for 30 minutes at 160° C. Upon cooling the reaction was diluted with CH₂Cl₂ (20 ml) and shaken with dil. HCl (3 ml). The organics were separated using a phase separator column with a NaSO₄ cartridge attached and concentrated. The oil was purified by silica chromatography (cyclohexane to 20% EtOAc in cyclohexane) to yield the acid. This was dissolved in methanol (5 ml) and 2M NaOH (1 ml) and was heated in a sealed vessel at 100° C. for 5 minutes using microwaves. Upon cooling, the mixture was concentrated in vacuo, the resulting residue diluted with water and extracted with EtOAc (×3), the organics dried (Na₂SO₄), filtered and concentrated to give the title compound (0.01 g).

LCMS rt=4.13 min [MH+] 386/388

### Example 77

### 3-[2-(5-Bromo-2-isobutyoxy-phenyl)-pyrrol-1-yl]-6-fluoro-benzoic acid

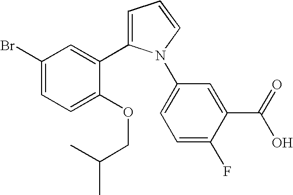

A solution of 1-(5-bromo-2-isobutyloxy-phenyl)-3-[1,3]dioxolan-2-yl-propan-1-one (0.1 g, 0.3 mmol), 3-amino-6-fluoro-benzoic acid (0.05 g, 0.3 mmol) and pTSA (cat) in anhydrous acetonitrile (1 mL) were heated in a microwave reactor for 30 minutes at 160° C. Upon cooling the reaction was diluted with CH₂Cl₂ (20 ml) and shaken with dil. HCl (3 ml). The organics were separated using a phase separator column with a NaSO₄ cartridge attached and concentrated. The oil was purified by silica chromatography (cyclohexane to 20% EtOAc in cyclohexane) to yield the title compound (0.01 g).

LCMS rt=4.20 min [MH+] 430/432

### 6-[2-(5-Chloro-2-methoxy-phenyl)-pyrrol-1-yl]-picolinic acid ethyl ester

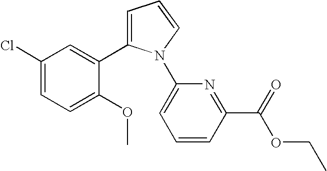

To a stirring solution of 2-bromo-6-[2-(5-chloro-2-methoxy-phenyl)-pyrrol-1-yl]-pyridine (0.16 g, 0.4 mmol) in anhydrous ethanol (1.3 mL) was added triethylamine (0.4 mL) and dichlorobis(triphenylphosphine) palladium (II) (5 mol %). The reaction was heated under a carbon monoxide atmosphere at 75° C. for sixty-five hours. The reaction was reduced in vacuo and purified by silica chromatography (cyclohexane to 25% EtOAc in cyclohexane) to yield a yellow oil (48 mg, 33%). LCMS t=3.58 min [MH⁺] 357/359

The following compound was prepared by a similar route to 6-[2-(5-chloro-2-methoxy-phenyl)-pyrrol-1-yl]-picolinic acid ethyl ester from the appropriate intermediates.

| Structure | Name | LCMS |
|---|---|---|
| 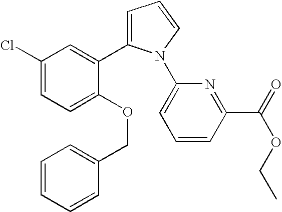 | 6-[2-(5-Chloro-2-benzyloxy- phenyl)-pyrrol-1-yl]-picolinic acid ethyl ester | t = 3.88 min [MH⁺] 433/435 |

### 6-[2-(5-Chloro-2-hydroxy-phenyl)-pyrrol-1-yl]-picolinic acid

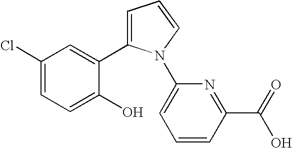

To a stirring solution of 6-[2-(5-chloro-2-methoxy-phenyl)-pyrrol-1-yl]-picolinic acid ethyl ester (47 mg, 0.132 mmol) in anhydrous DMF (2 mL) was added sodium thiomethoxide (0.66 mmol, 46 mg). The reaction was heated at 100° C. for four hours, allowed to cool, then partitioned between EtOAc (50 mL) and 2N HCl (30 mL). The aqueous layer was separated and extracted with EtOAc (2×50 mL). The organics were combined, dried (MgSO₄) and the solvent removed. The crude product was purified by silica chromatography (CH₂Cl₂ to 25% EtOAc in CH₂Cl₂) to yield a colourless glass (31 mg, 74%). LCMS t=3.62 min [MH⁺] 315/317.

### Example 78

### 6-}2-[5-Chloro-2-(2,4-difluoro-benzyloxy)-phenyl)-pyrrol-1-yl]-picolinic acid sodium salt

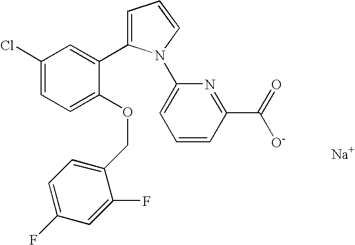

To a solution of 6-[2-(5-chloro-2-hydroxy-phenyl)-pyrrol-1-yl]-picolinic acid (0.098 mmol, 30.8 mg) in anhydrous methanol (2 mL) was added potassium carbonate (0.216 mmol, 30 mg) and potassium iodide (0.02 mmol, 3.2 mg). The reaction was heated to 60° C. before the dropwise addition of 2,4-difluorobenzyl bromide (0.196 mmol, 25 μL). The reaction was heated at 60° C. for a further 75 minutes. Solvent was removed from the reaction and the residue partitioned between 2N HCl (1 mL) and CH₂Cl₂ (2 mL). The organic layer was collected and the solvent removed. The sample was purified initially by mass directed HPLC, then via an aminopropyl SPE cartridge. The desired compound was dissolved in methanol/aqueous sodium hydroxide. The organic solvent was removed under a stream of nitrogen, and the resulting suspension extracted with CH₂Cl₂. The extracts were combined, dried, and the solvent removed to yield the desired product (22.4 mg, 49%).

LCMS t=3.82 min [MH⁺] 440/442.

### 3-[2-(5-Chloro-2-benzyloxy-phenyl)-5-chloro-pyrrol-1-yl]-benzoic acid ethyl ester

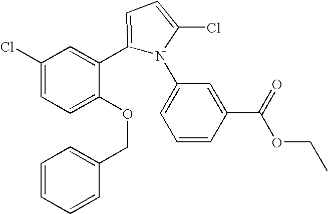

N-Chlorosuccinimide (26 mg, 0.20 mmol) was added to a stirred solution of 3-[2-(5-chloro-2-benzyloxy-phenyl)-5-pyrrol-1-yl]-benzoic acid ethyl ester (74 mg, 0.17 mmol) in THF (1.7 mL) at 0° C. Stirring was continued at 0° C. for 1 hour then allowed to warm slowly to room temperature and continued overnight. Water was added and the mixture extracted with DCM. The combined extracts were dried (Na₂SO₄) and and concentrated in vacuo to yield the title compound.

LCMS t=4.41 min [MH⁺] 466

The following compound was prepared by a similar route to 3-[2-(5-chloro-2-benzyloxy-phenyl)-5-chloro-pyrrol-1-yl]-benzoic acid ethyl ester from the appropriate intermediates.

| Structure | Name | LCMS |
|---|---|---|
| 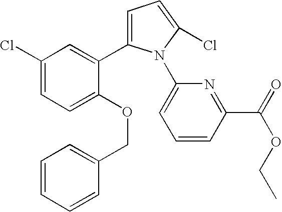 | 6-[2-(5-Chloro-2-benzyloxy- phenyl)-5-chloro-pyrrol-1-yl]- picolinic acid ethyl ester | t = 3.97 min [MH⁺]467/469/471 |

### Example 79

### 3-[2-(5-Chloro-2-benzyloxy-phenyl)-5-chloro-pyrrol-1-yl]-benzoic acid sodium salt

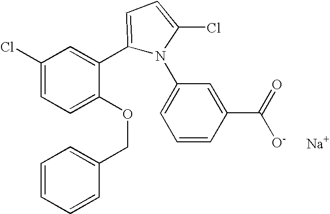

3-[2-(5-Chloro-2-benzyloxy-phenyl)-5-pyrrol-1-yl]-benzoic acid ethyl ester (80 mg) was dissolved in EtOH (1.8 mL) and 2M NaOH (0.9 mL) added. The mixture was stirred at room temperature for 4.5 hours then evaporated in vacuo. Water was added and the mixture extracted with EtOAc. The combined extracts were dried (Na₂SO₄) and concentrated in vacuo to yield the title compound.

LCMS t=4.09 min [MH⁺] 438; [MH⁻] 436, 438.

### Example 80

### 6-[2-(5-Chloro-2-benzyloxy-phenyl)-5-chloro-pyrrol-1-yl]-picolinic acid

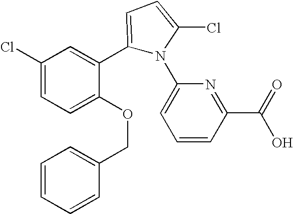

6-[2-(5-Chloro-2-benzyloxy-phenyl)-5-chloro-pyrrol-1-yl]-picolinic acid ethyl ester (12 mg) was dissolved in EtOH (2 mL) and 2M NaOH (1 mL) added. The mixture was stirred at room temperature for 18 hours then evaporated in vacuo. 2M HCl was added and the mixture extracted with EtOAc. The combined extracts were dried (Na₂SO₄) and concentrated in vacuo. The residue was purified using MDAP to yield the title compound.

LCMS t=4.28 min [MH⁺] 439/441/443

It is to be understood that the present invention covers all combinations of particular and preferred subgroups described herein above.

### ASSAYS FOR DETERMINING BIOLOGICAL ACTIVITY

The compounds of formula (I) can be tested using the following assays to demonstrate their prostanoid antagonist or agonist activity in vitro and in vivo and their selectivity. The prostaglandin receptors investigated are DP, EP₁, EP₂, EP₃, EP₄, FP, IP and TP.

The ability of compounds to antagonise EP₁, & EP₃ receptors may be demonstrated using a functional calcium mobilisation assay. Briefly, the antagonist properties of compounds are assessed by their ability to inhibit the mobilisation of intracellular calcium ([Ca²⁺]ᵢ) in response to activation of EP₁ or EP₃ receptors by the natural agonist hormone prostaglandin E₂ (PGE₂). Increasing concentrations of antagonist reduce the amount of calcium that a given concentration of PGE₂ can mobilise. The net effect is to displace the PGE₂ concentration-effect curve to higher concentrations of PGE₂. The amount of calcium produced is assessed using a calcium-sensitive fluorescent dye such as Fluo-3, AM and a suitable instrument such as a Fluorimetric Imaging Plate Reader (FLIPR). Increasing amounts of [Ca²⁺]ᵢ produced by receptor activation increase the amount of fluorescence produced by the dye and give rise to an increasing signal. The signal may be detected using the FLIPR instrument and the data generated may be analysed with suitable curve-fitting software.

The human EP₁ or EP₃ calcium mobilisation assay (hereafter referred to as ‘the calcium assay’) utilises Chinese hamster ovary-K1 (CHO-K1) cells into which a stable vector containing either EP₁ or EP₃ cDNA has previously been transfected. Cells are cultured in suitable flasks containing culture medium such as DMEM:F-12 supplemented with 10% v/v foetal calf serum, 2 mM L-glutamine, 0.25 mg/ml geneticin and 10 μg/ml puromycin.

For assay, cells are harvested using a proprietary reagent that dislodges cells such as Versene. Cells are re-suspended in a suitable quantity of fresh culture media for introduction into a 384-well plate. Following incubation for 24 hours at 37° C. the culture media is replaced with a medium containing fluo-3 and the detergent pluronic acid, and a further incubation takes place. Concentrations of compounds are then added to the plate in order to construct concentration-effect curves. This may be performed on the FLIPR in order to assess the agonist properties of the compounds. Concentrations of PGE₂ are then added to the plate in order to assess the antagonist properties of the compounds.

The data so generated may be analysed by means of a computerised curve-fitting routine. The concentration of compound that elicits a half-maximal inhibition of the calcium mobilisation induced by PGE₂ (pIC₅₀) may then be estimated.

Binding Assay for the Human Prostanoid EP₁ Receptor

Competition assay using [³H]-PGE2.

Compound potencies are determined using a radioligand binding assay. In this assay compound potencies are determined from their ability to compete with tritiated prostaglandin E₂ ([³H]-PGE₂) for binding to the human EP₁ receptor.

This assay utilises Chinese hamster ovary-K1 (CHO-K1) cells into which a stable vector containing the EP₁ cDNA has previously been transfected. Cells are cultured in suitable flasks containing culture medium such as DMEM:F-12 supplemented with 10% v/v foetal calf serum, 2 mM L-glutamine, 0.25 mg/ml geneticin, 10 μg/ml puromycin and 10 μM indomethacin.

Cells are detached from the culture flasks by incubation in calcium and magnesium free phosphate buffered saline containing 1 mM disodium ethylenediaminetetraacetic acid (Na₂EDTA) and 10 μM indomethacin for 5 min. The cells are isolated by centrifugation at 250×g for 5 mins and suspended in an ice cold buffer such as 50 mM Tris, 1 mM Na₂EDTA, 140 mM NaCl, 10 μM indomethacin (pH 7.4). The cells are homogenised using a Polytron tissue disrupter (2×10 burst at full setting), centrifuged at 48,000×g for 20 mins and the pellet containing the membrane fraction is washed three times by suspension and centrifugation at 48,000×g for 20 mins. The final membrane pellet is suspended in an assay buffer such as 10 mM 2-[N-morpholino]ethanesulphonic acid, 1 mM Na₂EDTA, 10 mM MgCl₂ (pH 6). Aliquots are frozen at −80° C. until required.

For the binding assay the cell membranes, competing compounds and [³H]-PGE₂ (3 nM final assay concentrabon) are incubated in a final volume of 100 μl for 30 min at 30° C. All reagents are prepared in assay buffer. Reactions are terminated by rapid vacuum filtration over GF/B filters using a Brandell cell harvester. The filters are washed with ice cold assay buffer, dried and the radioactivity retained on the filters is measured by liquid scintillation counting in Packard TopCount scintillation counter.

The data are analysed using non linear curve fitting techniques (GraphPad Prism 3) to determine the concentration of compound producing 50% inhibition of specific binding (IC₅₀).

By application of these techniques, compounds of the Examples had an antagonist pIC₅₀ value of 6.0 to 9.0 at EP₁ receptors and pIC₅₀ value of <6.0 at EP₃ receptors.

No toxicological effects are indicated/expected when a compound (of the invention) is administered in the above mentioned dosage range.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation the following claims:

What is claimed is:

1. A compound of formula (1): 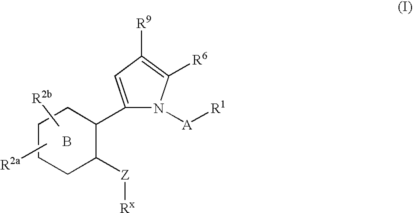
wherein A represents an optionally substituted aryl, or an optionally substituted 5- or 6-membered heterocyclyl ring, or an optionally substituted bicyclic heterocyclyl group;
B represents a phenyl or pyridyl ring;
Z represents O, S, SO, or SO₂;
R¹ represents CO₂R⁴, CN, CONR⁵R⁶, CH₂CO₂R⁴, OR⁴, optionally substituted alkyl, optionally substituted alkenyl, optionally substituted SO₂alkyl, SO₂NR⁵R⁶, NR⁵CONR⁵R⁶, COalkyl, 2H-tetrazol-5-yl-methyl, optionally substituted bicyclic heterocycle or optionally substituted heterocyclyl;
R^{2a} and R^{2b} each independently represents hydrogen, halogen, optionally substituted alkyl, optionally substituted alkoxy, CN, SO₂alkyl, SR⁵, NO₂, optionally substituted aryl, CONR⁵R⁶ or optionally substituted heteroaryl;
R^{x} represents optionally substituted alkyl wherein 1 or 2 of the non-terminal carbon atoms are optionally replaced by a group independently selected from NR⁴, O and SOₙ, wherein n is 0, 1 or 2: or R^{x} represents optionally substituted CQ^{a}Q^{b}-heterocyclyl optionally substituted CQ^{a}Q^{b}-bicyclic heterocyclyl or optionally substituted CQ^{a}Q^{b}-aryl;
R⁴ represents hydrogen or an optionally substituted alkyl;
R⁵ represents hydrogen or an optionally substituted alkyl;
R⁶ represents hydrogen or optionally substituted alkyl, optionally substituted heteroaryl, optionally substituted SO₂aryl, optionally substituted SO₂alkyl, optionally substituted SO₂heteroaryl CN, optionally substituted CQ^{a}Q^{b}aryl, optionally substituted CQ^{a}Q^{b}heteroaryl or COR⁷;
R⁷ represents hydrogen, optionally substituted alkyl, optionally substituted heteroaryl or optionally substituted aryl;
R⁸ represents hydrogen, Cl, CF₃, or C₁₋₃alkyl;
R⁹ represents halogen, hydrogen, CF₃, or C₁₋₃alkyl;
Q^{a} and Q^{b} are each independently selected from hydrogen and CH₃;
wherein when A is a 6-membered ring the R¹ substituent and pyrrole ring are attached to carbon atoms 1,2-, 1,3- or 1,4- relative to each other, and when A is a five-membered ring or bicyclic heterocyclyl group the R¹ substituent and phenyl ring are attached to substitutable carbon atoms 1,2- or 1,3- relative to each other;
and derivatives thereof.

2. A compound according to claim 1 wherein A is optionally substituted phenyl, optionally substituted pyridyl or optionally substituted isoquinolinyl.

3. (canceled)

4. A pharmaceutical composition comprising a compound according to claim 1 or a pharmaceutically acceptable derivative thereof together with a pharmaceutical carrier and/or excipient.

5. A compound according to claim 1 or a pharmaceutically acceptable derivative thereof for use as an active therapeutic substance.

6. A compound according to claim 1 or a pharmaceutically acceptable derivative thereof for use in the treatment of a condition which is mediated by the action of PGE₂ at EP₁ receptors.

7. A method of treating a human or animal subject suffering from a condition which is mediated by the action of PGE₂ at EP₁ receptors which comprises administering to said subject an effective amount of a compound according to claim 1 or a pharmaceutically acceptable derivative thereof.

8. A method of treating a human or animal subject suffering from inflammatory pain, neuropathic pain or visceral pain which method comprises administering to said subject an effective amount of a compound according to claim 1 or a pharmaceutically acceptable derivative thereof.

9-10. (canceled)
